(19) United States
(12) Patent Application Publication
(10) Pub. No.: US 2016/0271265 A1
(43) Pub. Date: Sep. 22, 2016

(54) INSULIN-LIKE GROWTH FACTOR MIMETICS FOR USE IN THERAPY

(71) Applicant: NATIONAL INSTITUTES OF HEALTH, Bethesda, MD (US)

(72) Inventors: Kenneth FISCHBECK, Bethesda, MD (US); David Jonathan GLASS, Cambridge, MA (US)

(73) Assignees: NOVARTIS AG, Basel (CH); NATIONAL INSTITUTES OF HEALTH, Bethesda, MD (US)

(21) Appl. No.: 15/026,792

(22) PCT Filed: Sep. 30, 2014
(86) PCT No.: PCT/IB2014/064952

§ 371 Date: Apr. 1, 2016

Related U.S. Application Data
(60) Provisional application No. 61/885,811, filed on Oct. 2, 2013.

(51) Int. Cl. A61K 47/48; A61K 38/18

(52) U.S. Cl. CPC A61K 47/48215; A61K 38/18

(57) ABSTRACT

This relates to the use of an IGF-1 mimetic in human therapy. In particular, disclosed herein is the use of an IGF-1 precursor protein, particularly a human IGF-1 precursor protein, comprising the E-peptide for the treatment of Spinal and Bulbar Muscular Atrophy (SBMA) in a patient suffering from said disease.

### TECHNICAL FIELD

This invention is in the field of Insulin-like growth factor 1 (IGF-1) modifications. In particular, it relates to modified IGF-1 polypeptides for use in therapy of Spinal and Bulbar Muscular Atrophy (SBMA).

### BACKGROUND

Spinal and bulbar muscular atrophy (SBMA) is a late-onset, X-linked, neurodegenerative disease characterized by progressive muscle weakness and atrophy, for which there is currently no effective disease-modifying therapy. The disease predominately affects adult males with an estimated prevalence of 1-2 per 100,000. SBMA is caused by an expansion of a trinucleotide CAG repeat encoding a polyglutamine tract within the first exon of the androgen receptor (AR) gene (Fischbeck K H (2012) Developing treatment for spinal and bulbar muscular atrophy. Prog Neurobiol; 99:257-61). The AR belongs to the nuclear hormone receptor superfamily and translocates to the nucleus upon androgen binding where it binds DNA, activating and repressing target genes. In SBMA, expansion of the polyglutamine tract in the AR confers a toxic gain of function in the mutant protein, resulting in its accumulation in the nuclei of motor neurons and other affected tissues. Some post-translational modifications modify the toxicity of the polyglutamine-expanded AR, likely by enhancing mutant AR clearance (Palazzolo I, Burnett B G, Young J E, et al (2007) Akt blocks ligand binding and protects against expanded polyglutamine androgen receptor toxicity. Hum Mol Gent; 16:1593-603).

While the major symptoms of SBMA are attributable to degeneration of spinal and brainstem lower motor neurons, sensory manifestations, signs of androgen insensitivity (gynecomastia and reduced fertility), and primary myopathic abnormalities also occur (Katsuno M, Tanaka F, Adachi H, et al (2012) Pathogenesis and therapy of spinal and bulbar muscular atrophy (SBMA). Prog Neurobiol; 99:246-56). Along these lines, muscle is very likely to be involved in the pathogenesis of SBMA. Muscle biopsies from SBMA patients exhibit myopathic as well as neurogenic changes, muscle pathology precedes motor neuron pathology in a knock-in mouse model of SBMA, and muscle specific overexpression of wild-type AR leads to an SBMA-like phenotype (Banno H, Katsuno M, Suzuki K, et al (2012) Pathogenesis and molecular targeted therapy of spinal and bulbar muscular atrophy (SBMA). Cell Tissue Res; 349:313-320).

SBMA transgenic mice overexpressing a muscle-specific isoform of IGF-1 in skeletal muscle showed increased Akt activation (Palazzolo I, Stack C, Kong L, et al (2009) Overexpression of IGF-1 in muscle attenuates disease in a mouse model of spinal and bulbar muscular atrophy. Neuron; 63:16-28), and increased phosphorylation and decreased aggregation of the AR protein. It has been shown that Akt activation effectively rescued behavioral and histopathological abnormalities, extended the life span, and reduced both muscle and spinal cord pathology of SBMA mice (Rinaldi C, Bott L C, Chen K L, et al (2012) Insulinlike growth factor (IGF)-1 administration ameliorates disease manifestations in a mouse model of spinal and bulbar muscular atrophy. Mol Med; 18:1261-8).

At present there are no effective treatments for SBMA. There is evidence that the toxicity of the mutant AR in SBMA is ligand dependent, although clinical trials of androgen-reducing agents have failed to produce clinically meaningful benefits (Fernandez-Rhodes L E, Kokkinis A D, White M J et al (2011) Efficacy and safety of dutaseride in patients with spinal and bulbar muscular atrophy: a randomized placebo-controlled trial. Lancet Neurol; 10:140-7, Katsuno M, Tanaka F, Adachi H, et al (2012) Pathogenesis and therapy of spinal and bulbar muscular atrophy (SBMA). Prog Neurobiol; 99:246-56).

Insulin-like growth factors (IGFs) are part of a complex system that cells use to communicate with their physiologic environment. This complex system (often referred to as the insulin-like growth factor axis) consists of two cell-surface receptors (IGF-1R and IGF-2R), two ligands (IGF-1 and IGF-2), a family of six high-affinity IGF-binding proteins (IGFBP 1-6), and associated IGFBP degrading enzymes (proteases). This system is important not only for the regulation of normal physiology but also for a number of pathological states (Glass, Nat Cell Biol 5:87-90, 2003).

Insulin-like growth factor-1 (IGF-1) is a powerful anabolic factor for skeletal muscle; its hypertrophic and anti-atrophic properties make it a biologically and clinically viable candidate to combat muscle wasting conditions that are associated with a decrease in endogenous IGF-1 (Clemmons D R (2007) Modifying IGF1 activity: an approach to treat endocrine disorders, atherosclerosis and cancer. Nat Rev Drug Discov; 6:821-37). In its mature form, human IGF-1, also called somatomedin, is a small protein of 70 amino acids that has been shown to stimulate growth of a wide range of cells in culture. The IGF-1 protein is initially encoded by three known splice variant mRNAs. The open reading frame of each mRNA encodes a precursor protein containing the 70 amino acid IGF-1 (SEQ ID NO:1) and a particular E-peptide at the C-terminus, depending on the particular IGF-1 mRNA. These E-peptides have been termed the Ea (rsvraqrhtdmpktqkevhlknasrgsagnknyrm; SEQ ID NO:2), Eb (rsvraqrhtdmpktqkyqppstnkntksqrrkgwpkthpggeqkegteaslqirgkkkeqrreigsrnaecrgkkgk; SEQ ID NO:3) and Ec (rsvraqrhtdmpktqkyqppstnkntksqrrkgstfeerk; SEQ ID NO:4) peptides and range from 35 to 87 amino acids in length and encompass a common sequence region at the N-terminus and a variable sequence region at the C-terminus. For example, the wild-type open reading frame for the IGF-1-Ea encodes a polypeptide of 135 amino acids including the leader sequence and a polypeptide of 105 amino acids without the leader sequence (gpetlcgaelvdalqfvcgdrgfyfnkptgygsssrrapqtgivdeccfrscdIrrlemycaplkpaksarsvraqrh tdmpktqkevhlknasrgsagnknyrm; SEQ ID NO:5). In physiological expression, the E-peptides are cleaved off of the precursor by endogenous proteases to yield the mature 70 amino acid IGF-1. However, the native IGF-1 protein has certain properties which may limit its efficacy. First, IGF-1 can be inactivated by cleavage at its receptor-binding site, which contains a dibasic motif that allows for rapid proteolysis when IGF-1 is incubated in serum. Second, IGF-1 can be inhibited by certain IGF-1 binding proteins, especially IGF Binding Protein 5 (IGFBP5), which has a higher affinity for IGF-1 than the hormone has for its receptor (IGF1R) (Clemmons D R (2012) Metabolic actions of insulin-like growth factor-1 in normal physiology and diabetes. Endocrinol Metab Clin North Am; 41:425-43). Finally, the mature form of IGF-1 is a small protein (7,600 Da) that is rapidly cleared from the circulation by renal filtration. These factors are thought to contribute to the lack of clinical efficacy in conditions of muscle wasting and short half-life of native IGF-1.

To address the pharmacokinetic and pharmacodynamic issues associated with IGF-1, a modified form of the native protein has been developed. The sequence of this human IGF-1 (hIGF-1) mimetic has been modified to increase its efficacy, by reducing proteolytic degradation, decreasing binding to inhibitory IGFBP5 and by adding a linear polyethylene glycol (PEG) chain at the N terminus (WO 2007/146689).

### SUMMARY OF THE INVENTION

Intervening in patients suffering from the neurodegenerative disease SBMA characterized by progressive muscle weakness and atrophy would be highly innovative and would meet a high unmet medical need. Indeed, this patient population currently has no therapeutic options. There is therefore a need to develop novel pharmaceutical compositions and methods to address neurodegenerative diseases caused by toxicity of the mutant AR and characterized by progressive muscle weakness and atrophy. This objective can be achieved by the methods and compositions provided within this disclosure.

The present invention relates to an IGF-1 mimetic, or a pharmaceutical composition comprising an IGF-1 mimetic, for use in therapy of Spinal and Bulbar Muscular Atrophy (SBMA) in a patient suffering from said disease.

In particular, the present invention relates to an IGF-1 mimetic, or a pharmaceutical composition comprising an IGF-1 mimetic, for use in therapy by preventing, ameliorating or reversing symptoms associated with SBMA in a patient suffering from SBMA.

In one embodiment of the invention, the IGF-1 mimetic, or the pharmaceutical composition comprising the IGF-1 mimetic, reduces or prevents degeneration of motor neurons in the brain stem and spinal cord of a patient suffering from SBMA.

In another embodiment of the invention, the IGF-1 mimetic, or the pharmaceutical composition comprising the IGF-1 mimetic, is used for preventing or reversing muscle weakness and/or atrophy in a patient suffering from SBMA.

In one embodiment of the invention, the IGF-1 mimetic, or the pharmaceutical composition comprising the IGF-1 mimetic, reduces mutant androgen receptor (AR) aggregation in skeletal muscle.

In another embodiment, the IGF-1 mimetic, or the pharmaceutical composition comprising the IGF-1 mimetic, reduces mutant androgen receptor (AR) aggregation in skeletal muscle and thus mutant AR toxicity. Hence, in one embodiment of the invention, the IGF-1 mimetic, or the pharmaceutical composition comprising the IGF-1 mimetic, is used for reducing mutant androgen receptor (AR) toxicity in skeletal muscle.

In a specific embodiment, the IGF-1 mimetic, or the pharmaceutical composition comprising the IGF-1 mimetic used in the inventive method is an IGF-1 mimetic which has been altered so as to avoid binding to inhibitory IGF1 binding proteins, and which has enhanced serum half-life, for example by virtue of being pegylated or mutated at specific positions as described in WO 2007/146689.

In a particular embodiment of the disclosure, the IGF-1 mimetic as such, or as part of a pharmaceutical composition, for use according to any one of the preceding embodiments, is a polypeptide comprising a human IGF-1 precursor protein comprising the E-peptide, particularly a precursor protein, which is modified such that the cleavage of the E-peptide from IGF-1 by a protease is reduced or avoided—compared to the unmodified IGF-1 protein—and/or which has a lower affinity to inhibitory IGF-1 binding proteins—compared to the unmodified IGF-1 protein-, in particular a lower affinity to the IGF-1 binding protein 5 (IGFBP5).

In a specific embodiment of the invention, the E-peptide is the Ea, Eb or Ec peptide, but particularly the Ea peptide.

At the N-terminus of the IGF-1 precursor protein, one, two or all of amino acids G1, P2, or E3 can be deleted or mutated. Further, R36 and R37 can be mutated to R36A and R37A, respectively.

In another related embodiment, the IGF-1 precursor protein comprises the Ea peptide comprising the following mutation: amino acid residues G1, P2, E3, R71 and S72 are deleted and R at position 37 is mutated to A and thus contains the following modification: ΔG1, ΔP2, ΔE3; R37A; ΔR71, ΔS72.

In an additional embodiment, the amino acids of the IGF-1 Ea-precursor protein G1, P2, E3, R37, R71 and S72 are deleted (IGF-1 Ea-peptide-ΔG1, ΔP2, ΔE3, ΔR37, ΔR71 and ΔS72.

In another specific embodiment, the arginine at position 37 of the IGF-1 precursor protein is mutated to alanine (R37A).

In a particular preferred embodiment, the IGF-1 Ea-precursor protein comprises the following mutations: amino acid residues E3, R71 and S72 are deleted and amino acid R at position 37 is mutated to alanine and, thus, comprises the following modification: ΔE3; R37A; ΔR71, ΔS72, wherein the numbering of the amino acids corresponds to SEQ ID NO: 5.

In a specific embodiment of the invention, the IGF-1 mimetic as such, or as part of a pharmaceutical composition, for use according to any one of the preceding embodiments, but particularly for use in therapy of Spinal and Bulbar Muscular Atrophy (SBMA) in a patient suffering from said disease, comprises or consists of the amino acid sequence as shown in SEQ ID NO: 6.

In certain other embodiments, the IGF-1 mimetic as such, or as part of a pharmaceutical composition, as described herein for use according to any one of the preceding embodiments, is pegylated. In particular, the IGF-1 mimetic comprises a poly(ethylene glycol) moiety, particularly consisting of a linear poly(ethylene glycol) chain, covalently attached to a side-chain of the precursor protein, particularly to the N-terminus.

In a specific embodiment, the present invention relates to a pegylated IGF-1 mimetic, or to a pharmaceutical composition comprising a pegylated IGF-1 mimetic, for use according to any one of the preceding embodiments, but particularly for use in therapy of Spinal and Bulbar Muscular Atrophy (SBMA) in a patient suffering from said disease, wherein said pegylated IGF-1 mimetic comprises the Ea-peptide wherein the amino acid residues E3, R71 and S72 are deleted and the amino acid R at position 37 mutated to A and thus contains the following modification: ΔE3; R37A; ΔR71, ΔS72.

In another specific embodiment, the present invention relates to a pegylated IGF-1 mimetic, or to a pharmaceutical composition comprising a pegylated IGF-1 mimetic, for use according to any one of the preceding embodiments, but particularly for use in therapy of Spinal and Bulbar Muscular Atrophy (SBMA) in a patient suffering from said disease, wherein said pegylated IGF-1 mimetic comprises or consists of the amino acid sequence as shown in SEQ ID NO: 6.

The invention also provides nucleic acid molecules that encode the IGF-1 mimetics of the invention as described herein for use in therapy of Spinal and Bulbar Muscular Atrophy (SBMA) in a patient suffering from said disease.

In particular, the invention provides a nucleic acid molecule encoding the amino acid sequence as shown in SEQ ID NO: 6, for use according to any one of the preceding embodiments, but particularly for use in therapy of Spinal and Bulbar Muscular Atrophy (SBMA) in a patient suffering from said disease. In a specific embodiment, this nucleic acid molecule exhibits the nucleotide sequence as shown in SEQ ID NO: 7.

In certain other embodiments, the pharmaceutical composition as described herein comprises the IGF-1 mimetic in a prophylactically or therapeutically effective amount and further a pharmaceutically acceptable carrier.

In particular, the IGF-1 mimetic as described herein, for use according to any of the above mentioned uses in therapy, is to be administered at a dose of 0.1 mg/kg-3 mg/kg body weight, particularly at a dose of about 0.1 mg/kg, about 0.3 mg/kg, about 1 mg/kg, about 2 mg/kg, about 3 mg/kg body weight, particularly in form of a single intravenous infusion.

Another aspect of the invention relates to a method of treating Spinal and Bulbar Muscular Atrophy (SBMA or Kennedy disease) in a patient suffering from said disease, the method comprising administering to said patient a therapeutically effective amount of the IGF-1 mimetic, or a pharmaceutical composition comprising a IGF-1 mimetic, as described herein.

In particular, the invention relates to a method for preventing, ameliorating or reversing symptoms associated with SBMA in a patient suffering from SBMA, the method comprising administering to said patient a therapeutically effective amount of the IGF-1 mimetic or a pharmaceutical composition comprising an IGF-1 mimetic in a therapeutically effective amount, as described herein. In one embodiment, said IGF-1 mimetic is an IGF-1 precursor protein, particularly a human IGF-1 precursor protein. In particular, the method according to the invention comprises administering of an IGF-1 precursor protein which is modified such that the cleavage of the E-peptide from IGF-1 by a protease is reduced. In certain embodiments of the invention, the IGF-1 precursor protein administered to said patients in a therapeutically effective amount comprises the Ea, Eb or Ec peptide, particularly the Ea peptide. In certain other embodiments of the disclosed method, the modification of the IGF-1 Ea-precursor protein administered to said patients in a therapeutically effective amount comprises deletion of the amino acid residues E3, R71, S72 and mutation or deletion of arginine at position 37, particularly the mutation R37A.

In a specific embodiment, the invention relates to a method for treating Spinal and Bulbar Muscular Atrophy (SBMA or Kennedy disease), or for preventing, ameliorating or reversing symptoms associated with SBMA, in a patient suffering from said disease, the method comprising administering to said patient a therapeutically effective amount of an IGF-1 precursor protein, or a pharmaceutical composition comprising an IGF-1 precursor protein in a therapeutically effective amount, wherein said IGF-1 precursor protein comprises the Ea peptide and the following modification: ΔE3; R37A; ΔR71; ΔS72, but particularly a precursor protein comprising the amino acid sequence as shown in SEQ ID NO: 6.

In particular, the IGF-1 Ea-precursor protein used in the above described method is pegylated and comprises the ΔE3; R37A; ΔR71; ΔS72 modifications. In particular said pegylated IGF-1 Ea precursor protein comprises the amino acid sequence as shown in SEQ ID NO: 6.

In particular, the IGF-1 Ea precursor protein used in the above described method comprises the ΔE3; R37A; ΔR71; ΔS72 modification (e.g. the precursor protein comprising the amino acid sequence as shown in SEQ ID NO: 6) and a poly(ethylene glycol) moiety, particularly a linear poly(ethylene glycol) moiety, covalently attached to an amino acid side-chain of the precursor protein.

In a particular embodiment of the disclosure the poly(ethylene glycol) moiety attached to the above described IGF-1 mimetics is linear poly(ethylene glycol) moiety having an overall molecular weight of from 20 to 100 kDa (kilo dalton). Consequently, in one embodiment of the disclosure the linear poly(ethylene glycol) moiety attached to the above described IGF-1 mimetics has an overall molecular weight of about 30 kDa.

In another aspect, the method according to the invention and as described herein further comprises reducing or preventing degeneration of motor neurons in the brain stem and spinal cord of a patient suffering from SBMA.

In still another aspect, the method according to the invention and as described herein further comprises preventing or reversing muscle weakness and/or atrophy in a patient suffering from SBMA and/or reducing mutant androgen receptor (AR) aggregation in skeletal muscle to reduce mutant AR toxicity.

The invention also relates to the use of the IGF-1 mimetic as described herein for the manufacture of a medicament for use in the treatment of Spinal and Bulbar Muscular Atrophy (SBMA or Kennedy disease), or for preventing, ameliorating or reversing symptoms associated with SBMA, in a patient suffering from said disease.

Embodiments of the disclosure are described in the following aspects:
1. An IGF-1 mimetic for use in therapy of Spinal and Bulbar Muscular Atrophy (SBMA) in a patient suffering from said disease, wherein the IGF-1 mimetic is a polypeptide comprising an IGF-1 precursor protein comprising the E-peptide.
2. The IGF-1 mimetic for use according to aspect 1 for preventing, ameliorating or reversing symptoms associated with SBMA in a patient suffering from SBMA.
3. The IGF-1 mimetic for use according to aspect 1 or aspect 2 for reducing or preventing degeneration of motor neurons in the brain stem and spinal cord of a patient suffering from SBMA.
4. The IGF-1 mimetic for use according to any one of the preceding aspects for preventing or reversing skeletal muscle weakness and/or atrophy in a patient suffering from SBMA.
5. The IGF-1 mimetic for use according to any one of the preceding aspects for reducing mutant androgen receptor (AR) aggregation in skeletal muscle to reduce mutant AR toxicity.
6. The IGF-1 mimetic for use according to any one of the preceding aspects, wherein the precursor protein is a human IGF-1 precursor protein.
7. The IGF-1 mimetic for use according to any one of the preceding aspects, wherein the precursor protein is modified such that the cleavage of the E-peptide from IGF-1 by a protease is reduced.
8. The IGF-1 mimetic for use according to any one of the preceding aspects, wherein the precursor protein comprises the Ea, Eb or Ec peptide.
9. The IGF-1 mimetic for use according to any one of the preceding aspects, wherein the precursor protein comprises the Ea peptide.
10. The IGF-1 Ea precursor mimetic for use according to any one of the preceding aspects, wherein the amino acid residues E3, R71 and S72 of the precursor protein are deleted.
11. The IGF-1 mimetic for use according to any one of the preceding aspects, wherein the arginine at position 37 of the precursor protein is mutated to an alanine (R37A).
12. The IGF-1 Ea precursor mimetic for use according to any one of the preceding aspects, wherein the precursor protein comprises the following modification: ΔE3; R37A; ΔR71, ΔS72.
13. The IGF-1 mimetic for use according to any one of the preceding aspects, wherein the precursor protein comprises the amino acid sequence as shown in SEQ ID NO: 6.
14. The IGF-1 mimetic for use according to any one of the preceding aspects further comprising a poly(ethylene glycol) moiety covalently attached to a side-chain of the precursor protein.
15. The IGF-1 mimetic for use according to aspect 14, wherein the pegylated precursor protein comprises the amino acid sequence as shown in SEQ ID NO: 6.
16. A pharmaceutical composition comprising the IGF-1 mimetic as recited in any one of aspects 6-15 for use in therapy of Spinal and Bulbar Muscular Atrophy (SBMA) in a patient suffering from said disease.
17. The composition of aspect 16 for use as recited in any one of aspects 1-5.
18. The composition for use according to aspect 16 or aspect 17, further comprising a pharmaceutically acceptable carrier.
19. The composition for use according to any one of aspects 16-18, comprising the IGF-1 mimetic as recited in any one of aspects 6-15 in a prophylactically or therapeutically effective amount.
20. The composition for use according to any one of aspects 16-19, wherein the IGF-1 mimetic is to be administered at a dose of 0.001-10 mg/kg body weight.
21. The composition for use according to aspect 20, wherein the IGF-1 mimetic is to be administered at a dose of about 0.01, about 00.3, about 0.1, about 0.3, about 0.5, about 1 mg/kg body weight.
22. The composition for use according to any one of aspects 16-21, wherein the IGF-1 mimetic is to be administered as a single intravenous infusion.
23. A method of treating Spinal and Bulbar Muscular Atrophy (SBMA or Kennedy disease) in a patient suffering from said disease as recited in any one of aspects 1-5, the method comprising administering to said patient a therapeutically effective amount of a the IGF-1 mimetic as recited in any one of aspects 6-15.
24. The use of the IGF-1 mimetic as recited in any one of aspects 6-15 for the manufacture of a medicament for use in the treatment of Spinal and Bulbar Muscular Atrophy (SBMA or Kennedy disease) in a patient suffering from said disease as recited in any one of aspects 1-5.

### DEFINITIONS

In order that the present disclosure may be more readily understood, certain terms are first defined. The technical terms and expressions used within the scope of this application are generally to be given the meaning commonly applied to them in the pertinent art if not otherwise indicated herein. Additional definitions are set forth throughout the detailed description.

As used in this specification and the appended claims, the singular forms “a”, “an”, and “the” include plural referents unless the context clearly dictates otherwise. Thus, for example, reference to “a compound” includes one or more compounds.

The term “comprising” means “including” e.g. a composition “comprising” X may consist exclusively of X or may include something additional e.g. X+Y.

The term “about” in relation to a numerical value x means, for example, x±10%.

The term “precursor” when used in the context of the present invention shall refer to the precursor of the mature human IGF-1 protein without signal peptide, but including the Ea, Eb or Ec peptide, respectively. The term “precursor” also refers to a human IGF-1 precursor protein comprising the mature 70 amino acid protein, or a IGF-1 mimetic of the identical or similar size, which is sufficient to bind the IGF1 Receptor—and the COOH terminal E-peptide which is often but not always cleaved from the mature region

A “patient” or “subject” for the purposes of the present invention is used interchangeably and meant to refer to humans. Thus, the methods are applicable human therapy.

The symbol “Δ” or the letters “d” or “D”: in the context of a protein description (e.g. “hIGF-1-Ea-Δ1-3, R37A, Δ 71-72) refers to an amino acid deletion. If not otherwise specified, the numbering of the specific amino acid positions corresponds to SEQ ID NO: 5. The single letter amino acid code refers to the following commonly used on letter code:

### The Single-Letter Amino Acid Code

G—Glycine (Gly); P—Proline (Pro); A—Alanine (Ala); V—Valine (Val); L—Leucine (Leu); I—Isoleucine (Ile); M—Methionine (Met); C—Cysteine (Cys); F—Phenylalanine (Phe); Y—Tyrosine (Tyr); W—Tryptophan (Trp); H—Histidine (His); K—Lysine (Lys); R—Arginine (Arg); Q—Glutamine (Gln); N—Asparagine (Asn); E—Glutamic Acid (Glu); D—Aspartic Acid (Asp); S—Serine (Ser); T—Threonine (Thr)

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease from occurring in a subject which may be predisposed to the disease; (b) inhibiting the disease, i.e. arresting its development: or (c) relieving the disease, i.e. causing regression of the disease. The terms “preventing”, “ameliorating” or “reversing” as used in the context of the present invention (treatment) refer to the prevention, amelioration or reversion of the disease conditions/pathogenesis observed in SBMA patients (e.g. see Katsuno M, Tanaka F, Adachi H, et al (2012) Pathogenesis and therapy of spinal and bulbar muscular atrophy (SBMA). Prog Neurobiol; 99:246-56). Consequently “preventing”, “ameliorating” or “reversing” inter alia refers to reduction of disease symptoms in a subject, i.e. increase in muscle mass and strength. The particular degree or level of such an increase may be in a range of at least 15%, 25%, 35%, 50%, 65%, 75%, 80%, 85%, 90%, 95%, 98% or more. The degree of prevention, amelioration or reversion may also be partial, such that the peculiarity of the disease conditions/pathogenesis in a patient is statistically significantly less pronounced than had the patient not received a composition of the present invention. Partial treatment results may be a decrease in severity of disease symptoms, an increase in frequency and duration of disease symptom-free periods, or a prevention of impairment or disability due to the disease affliction.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the patient being treated. Determination of a therapeutically effective amount is within the skill of the person skilled in the art. The therapeutically effective amount or dosage of a compound according to this invention can vary within wide limits and may be determined in a manner known in the relevant art. The dosage can vary within wide limits and will, of course, have to be adjusted to the individual requirements in each particular case.

The phrase “Insulin like growth factor 1 protein” refers to proteins being encoded by Insulin like growth factor 1 genes, particularly preferred is the human Insulin like growth factor 1 (hIGF-1) protein and variants thereof. An IGF-1 protein variant or an IGF-1 mimetic is a protein that differs by at least one amino acid from the IGF-1 wild-type sequence, wherein the term “wild-type sequence” refers to a polypeptide or gene sequence available in at least one naturally occurring organism or a polypeptide or gene sequence that has not been changed, mutated, or otherwise manipulated by man. The term IGF-1 variant and IGF-1 mimetic are used interchangeably throughout the document. An IGF-1 variant is also the IGF-1 precursor protein or the pro-IGF-1 protein comprising a peptide leader sequence. An IGF-1 variant can also be a fusion protein comprising an IGF-1 protein, e.g. a protein comprising an IGF-1 protein fused to a polyehtyleneglycol (PEG) moiety or a human IgG fc domain. Examples for IGF-1 variants are disclosed inter alia in the patent applications WO2007146689 (stabilized IGF-1 precursor proteins). An IGF-1 variant as described above retains its biological activity in the sense that such a protein can be considered as a functional equivalent of the wildtype IGF-1.

Functional equivalents with regard to the IGF-1 protein have to be understood as IGF-1 proteins comprising natural or artificial mutation. Mutations can be insertions, deletions or substitutions of one or more nucleic acids that do not diminish the biological activity of the IGF-1 protein. Functional equivalents having an identity of at least 80%, preferably 85%, more preferably 90%, most preferably more than 95%, very especially preferably at least 98% identity—but less than 100% identity to the IGF-1 wildtype protein, e.g. the human IGF-1 protein SEQ ID NO: 1. In case of fusion proteins, the % identity shall be defined only on the basis of the IGF-1 part of such a fusion protein.

### DETAILED DESCRIPTION OF THE INVENTION

Various aspects of the disclosure are described in further detail in the following subsections. At present there are no effective treatments for Spinal and Bulbar Muscular Atrophy (SBMA). Androgen-reducing agents have failed to produce clinically meaningful benefits (Fernandez-Rhodes L E, Kokkinis A D, White M J et al (2011) Efficacy and safety of dutaseride in patients with spinal and bulbar muscular atrophy: a randomized placebo-controlled trial. Lancet Neurol; 10:140-7, Katsuno M, Tanaka F, Adachi H, et al (2012). Pathogenesis and therapy of spinal and bulbar muscular atrophy (SBMA). Prog Neurobiol; 99:246-56).

Within the scope of the present invention, the means and methods are provided for effectively treating Spinal and Bulbar Muscular Atrophy (SBMA) in a patient suffering from said disease.

The invention relates to IGF-1 precursor polypeptides containing substantially an E-peptide that has been modified to prevent, reduce, or avoid the typical protease cleavage responsible for releasing the active IGF-1 from its E-peptides for use in therapy of Spinal and Bulbar Muscular Atrophy (SBMA) in a patient suffering from said disease.

Without being bound to any specific hypothesis, it is believed that IGF-1/Akt-mediated inhibition of mutant AR toxicity is an effective strategy to treat SBMA in vivo. The IGF-1 precursor polypeptide mimetics according to the invention have the potential to specifically reduce mutant AR toxicity in skeletal muscle, peripheral nerve, or other relevant cells, and thus to directly attenuate muscle degeneration and improve function in patients with SBMA.

IGF-1 treatment has only been tried for 1) treatment of IGF-1 deficiency; or 2) to make use of its insulin-like properties to supplant insulin therapy in diabetes; or 3) as a general anabolic due to its role as a second messenger of growth hormone. The preclinical/clinical work described in the prior art showed beneficial changes with IGF-1 treatment predominantly in muscle tissue which were associated with improved muscle function. The pathology of SBMA primarily involves the bulbar and spinal cord motor neurons. It has neither been demonstrated in the prior art that (i) treatment with IGF-1 mimetics could have an effect on diseases impacting spinal motor neurons, nor that (ii) IGF-1 mimetics can be used to drive Akt-mediated suppression of mutant AR toxicity.

Native IGF-1 does not have good drug-like properties. It is cleared very rapidly, and it is bound by inhibitory IGF1 binding proteins, preventing its efficacy. Consequently, native IGF-1 needs to be given at doses that cause a high Cmax, which risks cross-stimulation of the insulin receptor, resulting in hypoglycemia. Surprisingly, the disclosed IGF-1 mimetics, particularly those IGF-1 Ea-precursor proteins wherein one or more of amino acid residues E3, R71 or S72 are deleted and the arginine at position 37 of the precursor protein is mutated to alanine (R37A), have the potential to specifically reduce mutant AR toxicity in skeletal muscle, peripheral nerve, or other relevant cells, and thus to directly attenuate muscle degeneration and improve function in patients with SBMA, without causing the above mentioned problems. Such a precursor protein might comprise the amino acid sequence as shown in SEQ ID NO: 6. In another specific embodiment the above mentioned IGF-1 Ea precursor protein further comprises a poly(ethylene glycol) moiety covalently attached to a side-chain of the precursor protein. In a preferred embodiment, the poly(ethylene glycol) moiety is covalently attached to the N-terminus of the above described IGF-1 Ea precursor protein, e.g. a protein comprising the amino acids as depicted in sequence SEQ ID NO: 6.

In another preferred embodiment of the disclosure, the IGF-1 Ea precursor protein mimetic, or a pharmaceutical composition comprising said IGF-1 mimetic, for use in the treatment of a patient suffering from SBMA, comprises the amino acids as depicted in sequence SEQ ID NO: 6 and a linear poly(ethylene glycol) moiety having an overall molecular weight of about 30 kDa covalently attached to the N-terminus of said protein.

In furthermore preferred embodiment of the disclosure, the IGF-1 Ea precursor protein mimetic, or a pharmaceutical composition comprising said IGF-1 mimetic, for use in the treatment of a patient suffering from SBMA, consists of the amino acids as depicted in sequence SEQ ID NO: 6 and comprises linear a poly(ethylene glycol) moiety having an overall molecular weight of about 30 kDa covalently attached to the N-terminus of said protein.

Another preferred embodiment of the disclosure relates to a method of Spinal and Bulbar Muscular Atrophy (SBMA or Kennedy disease) treatment in a patient suffering from said disease, the method comprising administering to said patient a therapeutically effective amount of an IGF-1 precursor protein mimetic, or a pharmaceutical composition comprising said IGF-1 precursor protein mimetic in a therapeutically effective amount, wherein said IGF-1 precursor protein comprises the amino acids as depicted in sequence SEQ ID NO: 6 and a linear poly(ethylene glycol) moiety having an overall molecular weight of about 30 kDa covalently attached to the N-terminus of said protein.

In another preferred embodiment of the disclosure, relates to a method of Spinal and Bulbar Muscular Atrophy (SBMA or Kennedy disease) treatment in a patient suffering from said disease, the method comprising administering to said patient a therapeutically effective amount of a IGF-1 precursor protein mimetic, or a pharmaceutical composition comprising said IGF-1 precursor protein mimetic in a therapeutically effective amount, wherein said IGF-1 precursor protein consists of the amino acids as depicted in sequence SEQ ID NO: 6 and comprises linear a poly(ethylene glycol) moiety having an overall molecular weight of about 30 kDa covalently attached to the N-terminus of said protein.

### Screening for Active IGF Precursor Polypeptides

The usefulness of any of the polypeptides of the invention can be assessed by using the assays disclosed in WO 2007/146689 (see, for example, pages 8-14) including stability testing, AKT phosphorylation assay, IGF-1 receptor specificity determination, in vivo testing in mouse models of hypertrophy, in vivo testing in muscle atrophy models, the content of which is incorporated herein by reference.

### Critical Mutations:

It was shown in WO 2007/146689 that the IGF precursor polypeptide that contains substantially its E-peptide remains bioactive and stable in the presence of serum. To ensure that the E-peptide is not cleaved by endogenous proteases targeting the dibasic protease site, in general either of the two N-terminal dibasic amino acids of the E-peptide in the precursor is deleted, mutated, or otherwise masked. In the case of hIGF-1, these two amino acids are R71 and S72.

### Mutations at the N-Terminus of Mature IGF:

In certain embodiments of the invention, the IGF precursor polypeptides have deletions or mutations of the first few N-terminal amino acids. In the case of IGF-1, any of the first three N-terminal amino acids can be deleted or mutated, either alone or in combination

Further particulars on alternative mutation sites and on modifications that enable the prevention of cleavage of the E-peptide by endogenous proteases are provided in WO 2007/146689 (see, for example, pages 14 and 15), the content of which is incorporated herein by reference.

Strategies to increase the half-life of IGF-1 have been described in the prior art. Strategies that have been contemplated are

(i) the production of IGF-1 variants comprising specific mutations aiming to prevent the cleavage of IGF-1 in human serum by serine proteases, or to alleviate the negative impact of IGF-1 binding proteins on the availability or serum half-life of IGF-1 (WO200040613, WO05033134, WO2006074390, WO2007/146689);
(ii) the production of IGF-1 fusion proteins, wherein the mature IGF-1 protein is fused to a human immunoglobulin Fc region (WO2005033134, WO200040613);
(iii) the use of IGF-1 precursor proteins wherein cleavage of the E-peptide from IGF-1 by a protease is reduced by modification of the precursor protein (WO2007146689);
(iv) combinations of the above described strategies ((i)/(ii) WO05033134, (i)/(ii) WO200040613, (i)/(iii) WO2007146689).

Hence, in addition to the herein described hIGF-1-Ea precursor polypeptide variants, the following additional protein variants can be produced and used in accordance with the invention in the therapy of Spinal and Bulbar Muscular Atrophy (SBMA) in a patient suffering from said disease:
a) The IGF-1 variants described in WO2006066891, characterized in that said IGF-1 variant has been mutated at up to three amino acid at positions 27, 37, 65, 68 of the wild-type IGF-I amino acid sequence.
b) The IGF-1 variants described in WO 2008025528, wherein said fusion proteins comprise amino acid substitutions at positions lysine 27, 65 and/or 68.
c) The IGF-1 variants described in WO200040613, in particular a fusion polypeptide, comprising (a) a human IGF1 variant polypeptide of SEQ ID NO: 1 with the deletions of amino acid residues 1-3, 37, and 65-70; and (b) a human IgG fc domain.

In addition to the above described hIGF-1-Ea precursor polypeptides variants, the following additional protein variants can be produced and used in accordance with the invention in the therapy of Spinal and Bulbar Muscular Atrophy (SBMA) in a patient suffering from said disease:

(1) E3 is deleted, amino acid R36 is substituted by glutamine (Q) and the amino acids R71 and S72 are deleted.

(SEQ ID NO: 8)
gptlcgaelvdalgfvcgdrgfyfnkptgygsssgrapgtgivdeccfrs
cdlrrlemycaplkpaksavragrhtdmpktgkevhlknasrgsagnkny
rm.

(2) E3 is deleted, amino acid R37 is substituted by glutamic acid (E) and the amino acids R71 and S72 are deleted.

(SEQ ID NO: 9)
gptlcgaelvdalqfvcgdrgfyfnkptgygsssreapqtgivdeccfrs
cdlrrlemycaplkpaksavraqrhtdmpktqkevhlknasrgsagnkny
rm.

(3) E3 is deleted, amino acid R37 is substituted by alanine and the amino acids R71 and S72 are deleted.

(SEQ ID NO: 10)
gptlcgaelvdalqfvcgdrgfyfnkptgygsssraapqtgivdeccfrs
cdlrrlemycaplkpaksavraqrhtdmpktqkevhlknasrgsagnkny
rm.

(4) E3 is deleted, amino acid R37 is substituted by proline (P) and the amino acids R71 and S72 are deleted.

(SEQ ID NO: 11)
gptlcgaelvdalqfvcgdrgfyfnkptgygsssrpapqtgivdeccfrs
cdlrrlemycaplkpaksavraqrhtdmpktqkevhlknasrgsagnkny
rm.

(5) E3 is deleted, amino acid R36 and R37 are both substituted by glutamine (Q) and the amino acids R71 and S72 are deleted.

(SEQ ID NO: 12)
gptlcgaelvdalqfvcgdrgfyfnkptgygsssqqapqtgivdeccfrs
cdlrrlemycaplkpaksavraqrhtdmpktqkevhlknasrgsagnkny
rm.

(6) E3 is deleted, amino acid R36 is substituted by glutamine (Q), R37 is substituted by alanine and the amino acids R71 and S72 are deleted.

(SEQ ID NO: 13)
gptlcgaelvdalqfvcgdrgfyfnkptgygsssqaapqtgivdeccfrs
cdlrrlemycaplkpaksavraqrhtdmpktqkevhlknasrgsagnkny
rm.

(7) E3 is deleted, amino acid R36 is substituted by glutamine (Q) and the amino acids R71 and S72 are deleted and amino acid R74 is mutated to glutamine (Q).

(SEQ ID NO: 14)
gptlcgaelvdalqfvcgdrgfyfnkptgygsssqrapqtgivdeccfrs
cdlrrlemycaplkpaksavqaqrhtdmpktqkevh1knasrgsagnkny
rm.

(8) E3 is deleted, amino acid R36 is substituted by glutamine (Q) and the amino acids R71 and S72 are deleted and amino acids R74 and R77 are mutated to glutamine (Q).

(SEQ ID NO: 15)
gptlcgaelydalqfycgdrgfyfnkptgygsssqrapqtgiydeccfrs
cdlrrlemycaplkpaksayqaqqhtdmpktqkeyhlknasrgsagnkny
rm.

(9) E3 is deleted, amino acid R36 is substituted by glutamine (Q) and the amino acids R71 and S72 are deleted and amino acids R74, R77 and R104 are mutated to glutamine (Q).

(SEQ ID NO: 16)
gptlcgaelvdalqfvcgdrgfyfnkptgygsssqrapqtgivdeccfrs
cdlrrlemycaplkpaksavqaqqhtdmpktqkevhlknasrgsagnkny
qm.

(10) E3 is deleted, amino acid R37 is substituted by glutamic acid (E) and the amino acids R71 and S72 are deleted and amino acid R77 is mutated to glutamine (Q).

(SEQ ID NO: 17)
gptlcgaelvdalqfvcgdrgfyfnkptgygsssreapqtgivdeccfrs
cdlrrlemycaplkpaksavraqqhtdmpktqkevhlknasrgsagnkny
rm.

(11) E3 is deleted, amino acid R37 is substituted by glutamic acid (E) and the amino acids R71 and S72 are deleted and amino acids R74 and R77 are mutated to glutamine (Q).

(SEQ ID NO: 18)
gpticgaelvdalqfvcgdrgfyfnkptgygsssreapqtgivdeccfrs
cdlrrlemycaplkpaksavqaqqhtdmpktqkevhlknasrgsagnkny
rm.

(12) E3 is deleted, amino acid R37 is substituted by glutamic acid (E) and the amino acids R71 and S72 are deleted and amino acids R74, R77 and R104 are mutated to glutamine (Q).

(SEQ ID NO: 19)
gptlcgaelvdalqfvcgdrgfyfnkptgygsssreapqtgivdeccfrs
cdlrrlemycaplkpaksavqaqqhtdmpktqkevhlknasrgsagnkny
qm.

(13) E3 is deleted, amino acid R37 is substituted by alanine (A) and the amino acids R71 and S72 are deleted and amino acid R74 is mutated to glutamine (Q).

(SEQ ID NO: 20)
gptlcgaelvdalqfvcgdrgfyfnkptgygsssraapqtgivdeccfrs
cdlrrlemycaplkpaksavqaqrhtdmpktqkevhlknasrgsagnkny
rm.

(14) E3 is deleted, amino acid R37 is substituted by alanine (A) and the amino acids R71 and S72 are deleted and amino acids R74 and R77 are mutated to glutamine (Q).

(SEQ ID NO: 21)
gptlcgaelydalqfycgdrgfyfnkptgygsssraapqtgiydeccfrs
cdlrrlemycaplkpaksayqaqqhtdmpktqkeyhlknasrgsagnkny
rm.

(15) E3 is deleted, amino acid R37 is substituted by alanine (A) and the amino acids R71 and S72 are deleted and amino acids R74, R77 and R104 are mutated to glutamine (Q).

(SEQ ID NO: 22)
gptlcgaelvdalqfvcgdrgfyfnkptgygsssraapqtgivdeccfrs
cdlrrlemycaplkpaksavqaqqhtdmpktqkevhlknasrgsagnkny
qm.

(16) E3 is deleted, amino acid R37 is substituted by proline (P) and the amino acids R71 and S72 are deleted and amino acid R74 is mutated to glutamine (Q).

(SEQ ID NO: 23)
gptlcgaelvdalqfvcgdrgfyfnkptgygsssrpapqtgivdeccfrs
cdlrrlemycaplkpaksavqaqrhtdmpktqkevhlknasrgsagnkny
rm.

(17) E3 is deleted, amino acid R37 is substituted by proline (P) and the amino acids R71 and S72 are deleted and amino acids R74 and R77 are mutated to glutamine (Q).

(SEQ ID NO: 24)
gptlcgaelydalqfycgdrgfyfnkptgygsssrpapqtgiydeccfrs
cdlrrlemycaplkpaksayqaqqhtdmpktqkeyhlknasrgsagnkny
rm.

(18) E3 is deleted, amino acid R37 is substituted by proline (P) and the amino acids R71 and S72 are deleted and amino acids R74, R77 and R104 are mutated to glutamine (Q).

(SEQ ID NO: 25)
gptlcgaelvdalqfvcgdrgfyfnkptgygsssrpapqtgivde
ccfrscdlrrlemycaplkpaksavqaqqhtdmpktqkevhlkna
srgsagnknyqm.

(19) E3 is deleted, amino acid R36 and R37 are both substituted by glutamine (Q) and the amino acids R71 and S72 are deleted and amino acid R74 is mutated to glutamine (Q).

(SEQ ID NO: 26)
gptlcgaelvdalqfvcgdrgfyfnkptgygsssqqapqtgivde
ccfrscdlrrlemycaplkpaksavqaqrhtdmpktqkevhlkna
srgsagnknyrm.

(20) E3 is deleted, amino acid R36 and R37 are both substituted by glutamine (Q) and the amino acids R71 and S72 are deleted and amino acids R74 and R77 are mutated to glutamine (Q).

(SEQ ID NO: 27)
gptlcgaelvdalqfvcgdrgfyfnkptgygsssqqapqtgivde
ccfrscdlrrlemycaplkpaksavqaqqhtdmpktqkevhlkna
srgsagnknyrm.

(21) E3 is deleted, amino acid R36 is substituted by glutamine (Q), R37 is substituted by alanine and the amino acids R71 and S72 are deleted and amino acids R74 and R77 are mutated to glutamine (Q).

(SEQ ID NO: 28)
gptlcgaelvdalqfvcgdrgfyfnkptgygsssqaapqtgivde
ccfrscdlrrlemycaplkpaksavqaqqhtdmpktqkevhlkna
srgsagnknyrm.

(22) E3 is deleted, amino acid R36 and R37 are both substituted by glutamine (Q) and the amino acids R71 and S72 are deleted and amino acids R74, R77 and R104 are mutated to glutamine (Q).

(SEQ ID NO: 29)
gptlcgaelvdalqfvcgdrgfyfnkptgygsssqqapqtgivde
ccfrscdlrrlemycaplkpaksavqaqqhtdmpktqkevhlkna
srgsagnknyqm.

(23) E3 is deleted, amino acid R36 is substituted by glutamine (Q), R37 is substituted by alanine and the amino acids R71 and S72 are deleted and amino acids R74, R77 and R104 are mutated to glutamine (Q).

(SEQ ID NO: 30)
gptlcgaelydalqfycgdrgfyfnkptgygsssqaapqtgiyde
ccfrscdlrrlemycaplkpaksayqaqqhtdmpktqkeyhlkna
srgsagnknyqm.

(24) E3 is deleted, amino acid R36 is substituted by glutamine (Q) and the amino acids K68, S69, A70, R71 and S72 are deleted.

(SEQ ID NO: 31)
gptlcgaelvdalqfvcgdrgfyfnkptgygsssqrapqtgivde
ccfrscdlrrlemycaplkpavraqrhtdmpktqkevhlknasrg
sagnknyrm.

(25) E3 is deleted, amino acid R37 is substituted by glutamic acid (E) and the amino acids K68, S69, A70, R71 and S72 are deleted.

(SEQ ID NO: 32)
gptlcgaelvdalqfvcgdrgfyfnkptgygsssreapqtgivde
ccfrscdlrrlemycaplkpavraqrhtdmpktqkevhlknasrg
sagnknyrm.

(26) E3 is deleted, amino acid R37 is substituted by alanine and the amino acids K68, S69, A70. R71 and S72 are deleted.

(SEQ ID NO: 33)
gptlcgaelvdalgfvcgdrgfyfnkptgygsssraapgtgivde
ccfrscdlrrlemycaplkpavragrhtdmpktgkevhlknasrg
sagnknyrm.

(27) E3 is deleted, amino acid R37 is substituted by proline (P) and the amino acids K68, S69, A70, R71 and S72 are deleted.

(SEQ ID NO: 34)
gptlcgaelvdalqfvcgdrgfyfnkptgygsssrpapqtgivde
ccfrscdlrrlemycaplkpavraqrhtdmpktqkevhlknasrg
sagnknyrm.

(28) E3 is deleted, amino acid R36 and R37 are both substituted by glutamine (Q) and the amino acids K68, S69, A70, R71 and S72 are deleted.

(SEQ ID NO: 35)
gptlcgaelvdalqfvcgdrgfyfnkptgygsssqqapqtgivde
ccfrscdlrrlemycaplkpaqvraqrhtdmpktqkevhlknasr
gsagnknyrm.

(29) E3 is deleted, amino acid R36 is substituted by glutamine (Q), R37 is substituted by alanine and the amino acids K68, S69, A70, R71 and S72 are deleted.

(SEQ ID NO: 36)
gptlcgaelvdalqfvcgdrgfyfnkptgygsssqaapqtgivde
ccfrscdlrrlemycaplkpavraqrhtdmpktqkevhlknasrg
sagnknyrm.

(30) E3 is deleted, amino acid R36 is substituted by glutamine (Q) and the amino acids K68, S69, A70, R71 and S72 are deleted and amino acid R74 is mutated to glutamine (Q).

(SEQ ID NO: 37)
gptlcgaelvdalqfvcgdrgfyfnkptgygsssqrapqtgivde
ccfrscdlrrlemycaplkpavqaqrhtdmpktqkevhlknasrg
sagnknyrm.

(31) E3 is deleted, amino acid R36 is substituted by glutamine (Q) and the amino acids K68, S69, A70, R71 and S72 are deleted and amino acids R74 and R77 are mutated to glutamine (Q).

(SEQ ID NO: 38)
gptlcgaelydalqfycgdrgfyfnkptgygsssqrapqtgiyde
ccfrscdlrrlemycaplkpayqaqqhtdmpktqkeyhlknasrg
sagnknyrm.

(32) E3 is deleted, amino acid R36 is substituted by glutamine (Q) and the amino acids K68, S69, A70, R71 and S72 are deleted and amino acids R74, R77 and R104 are mutated to glutamine (Q).

(SEQ ID NO: 39)
gptlcgaelydalqfycgdrgfyfnkptgygsssqrapqtgiyde
ccfrscdlrrlemycaplkpayqaqqhtdmpktqkeyhlknasrg
sagnknyqm.

(33) E3 is deleted, amino acid R37 is substituted by glutamic acid (E) and the amino acids K68, S69, A70, R71 and S72 are deleted and amino acid R74 is mutated to glutamine (Q).

(SEQ ID NO: 40)
gptlcgaelvdalqfvcgdrgfyfnkptgygsssreapqtgivde
ccfrscdlrrlemycaplkpavqaqrhtdmpktqkevhlknasrg
sagnknyrm.

(34) E3 is deleted, amino acid R37 is substituted by glutamic acid (E) and the amino acids K68, S69, A70, R71 and S72 are deleted and amino acids R74 and R77 are mutated to glutamine (Q).

(SEQ ID NO: 41)
gptlcgaelydalgfycgdrgfyfnkptgygsssreapgtgiyde
ccfrscdlrrlemycaplkpaygagghtdmpktgkeyhlknasrg
sagnknyrm.

(35) E3 is deleted, amino acid R37 is substituted by glutamic acid (E) and the amino acids K68, S69, A70, R71 and S72 are deleted and amino acids R74, R77 and R104 are mutated to glutamine (Q).

(SEQ ID NO: 42)
gptlcgaelvdalqfvcgdrgfyfnkptgygsssreapqtgivde
ccfrscdlrrlemycaplkpavqaqqhtdmpktqkevhlknasrg
sagnknyqm.

(36) E3 is deleted, amino acid R37 is substituted by alanine (A) and the amino acids K68, S69, A70, R71 and S72 are deleted and amino acid R74 is mutated to glutamine (Q).

(SEQ ID NO: 43)
gptlcgaelvdalqfvcgdrgfyfnkptgygsssraapqtgivde
ccfrscdlrrlemycaplkpavqaqrhtdmpktqkevhlknasrg
sagnknyrm.

(37) E3 is deleted, amino acid R37 is substituted by alanine (A) and the amino acids K68, S69, A70, R71 and S72 are deleted and amino acids R74 and R77 are mutated to glutamine (Q).

(SEQ ID NO: 44)
gptlcgaelvdalqfvcgdrgfyfnkptgygsssraapqtgivde
ccfrscdlrrlemycaplkpavqaqqhtdmpktqkevhlknasrg
sagnknyrm.

(38) E3 is deleted, amino acid R37 is substituted by alanine (A) and the amino acids K68, S69, A70, R71 and S72 are deleted and amino acids R74, R77 and R104 are mutated to glutamine

(SEQ ID NO: 45)
gptlcgaelvdalqfvcgdrgfyfnkptgygsssraapqtgivde
ccfrscdlrrlemycaplkpavqaqqhtdmpktqkevhlknasrg
sagnknyqm.

(39) E3 is deleted, amino acid R37 is substituted by proline (P) and the amino acids K68, S69, A70, R71 and S72 are deleted and amino acid R74 is mutated to glutamine (Q).

(SEQ ID NO: 46)
gptlcgaelvdalqfvcgdrgfyfnkptgygsssrpapqtgivde
ccfrscdlrrlemycaplkpavqaqrhtdmpktqkevhlknasrg
sagnknyrm.

(40) E3 is deleted, amino acid R37 is substituted by proline (P) and the amino acids K68, S69, A70, R71 and S72 72 are deleted and amino acids R74 and R77 are mutated to glutamine (Q).

(SEQ ID NO: 47)
gptlcgaelydalqfycgdrgfyfnkptgygsssrpapqtgiyde
ccfrscdlrrlemycaplkpayqaqqhtdmpktqkeyhlknasrg
sagnknyrm.

(41) E3 is deleted, amino acid R37 is substituted by proline (P) and the amino acids K68, S69, A70, R71 and S72 are deleted and amino acids R74, R77 and R104 are mutated to glutamine (Q).

(SEQ ID NO: 48)
gptlcgaelydalqfycgdrgfyfnkptgygsssrpapqtgiyde
ccfrscdlrrlemycaplkpayqaqqhtdmpktqkeyhlknasrg
sagnknyqm.

(42) E3 is deleted, amino acid R36 and R37 are both substituted by glutamine (Q) and the amino acids K68, S69, A70, R71 and S72 are deleted and amino acid R74 is mutated to glutamine (Q).

(SEQ ID NO: 49)
gptlcgaelvdalqfvcgdrgfyfnkptgygsssqqapqtgivde
ccfrscdlrrlemycaplkpavqaqrhtdmpktqkevhlknasrg
sagnknyrm.

(43) E3 is deleted, amino acid R36 and R37 are both substituted by glutamine (Q) and the amino acids K68, S69, A70, R71 and S72 are deleted and amino acids R74 and R77 are mutated to glutamine (Q).

(SEQ ID NO: 50)
gptlcgaelvdalqfvcgdrgfyfnkptgygsssqqapqtgivde
ccfrscdlrrlemycaplkpavqaqqhtdmpktqkevhlknasrg
sagnknyrm.

(44) E3 is deleted, amino acid R36 is substituted by glutamine (Q), R37 is substituted by alanine and the amino acids K68, S69, A70, R71 and S72 are deleted and amino acids R74 and R77 are mutated to glutamine (Q).

(SEQ ID NO: 51)
gptlcgaelvdalqfvcgdrgfyfnkptgygsssqaapqtgivde
ccfrscdlrrlemycaplkpavqaqqhtdmpktqkevhlknasrg
sagnknyrm.

(45) E3 is deleted, amino acid R36 and R37 are both substituted by glutamine (Q) and the amino acids K68, S69, A70, R71 and S72 are deleted and amino acids R74, R77 and R104 are mutated to glutamine (Q).

(SEQ ID NO: 52)
gptlcgaelvdalqfvcgdrgfyfnkptgygsssqqapqtgivde
ccfrscdlrrlemycaplkpavqaqqhtdmpktqkevhlknasrg
sagnknyqm.

(46) E3 is deleted, amino acid R36 is substituted by glutamine (Q), R37 is substituted by alanine and the amino acids K68, S69, A70, R71 and S72 are deleted and amino acids R74, R77 and R104 are mutated to glutamine (Q).

(SEQ ID NO: 53)
gptlcgaelydalqfycgdrgfyfnkptgygsssqaapqtgiyde
ccfrscdlrrlemycaplkpayqaqqhtdmpktqkeyhlknasrg
sagnknyqm.

In another embodiment the disclosure relates to the use of the above described proteins (e.g. as depicted in SEQ ID NOs: 6 and 8-53), in the therapy of Spinal and Bulbar Muscular Atrophy (SBMA) in a patient suffering from said disease, wherein said molecules instead of being mutated at the positions 3, comprise a deletion of the amino acids 1-3.

Furthermore, in another embodiment the disclosure relates to the use of the above described proteins (e.g. as depicted in SEQ ID NOs: 6 and 8-53), in the therapy of Spinal and Bulbar Muscular Atrophy (SBMA) in a patient suffering from said disease, wherein the amino acid G42 is deleted or substituted by the amino acid serine.

### Use of Glycosylation:

The in vivo half-life of the polypeptides of the invention can be improved by the addition of N-linked glycosylation sites into either the IGF or the E-peptide portions of the precursor when expressed in mammalian or other eukaryotic cells capable of N-linked glycosylation. It has been shown in vitro that human IGF-1 Ea is glycosylated at N92 and N100, as these portions of Ea fits the consensus N-linked glycosylation sequence of N-X-S/T, where X can be any amino acid and the third amino acid of the triplet is either S or T. It is also know that the adjacent amino acid context of the consensus will affect how strongly the asparagine is glycosylated. Therefore, one strategy to introduce a glycosylation site into Eb or Ec is to insert Ea amino acids around the consensus sequence into roughly the same part of Eb or Ec. A particular implementation of this strategy is illustrated in the Examples disclosed in WO 2007/146689. In any event, any other consensus N-linked glycosylation site, including surrounding context amino acids, known to the skilled artisan can be inserted into a precursor polypeptide of the invention. In addition, O-linked glycosylation of a polypeptide of the invention can be accomplished by choosing the particular host used for production of the polypeptide. For example, use of certain yeast strains for IGF-1 expression results in the addition of oligosaccharides on a serine or threonine. See e.g., U.S. Pat. No. 5,273,966.

### Addition of Poly(Ethylene Glycol):

To address the pharmacokinetic and pharmacodynamic issues associated with IGF-1, a modified form of the native protein has been developed. The sequence of this human IGF-1 (hIGF-1) mimetic has been modified to increase its efficacy, by reducing proteolytic degradation, decreasing binding to inhibitory IGFBP5 and by adding a linear polyethylene glycol (PEG) chain at the N terminus (WO 2007/146689).

The preparation of PEGylated version of IGF-1 variants for the treatment of neuromuscular disorders was also described in WO2008025528, WO2009121759 A2 and WO2006066891. Usually PEG is attached to amino groups of the protein. However, a major limitation of this amino pegylation approach is that proteins typically contain a considerable amount of lysine residues and therefore the poly(ethylene glycol) groups are attached to the protein in a non-specific manner. Pegylation of amino residue required for biological activity (e.g. residues near or at the active site of the protein) can result in low specific activity or inactivation of the protein. To avoid some of the above described drawbacks WO2006066891 describes the use of conjugates consisting of IGF-1 variants and one or two poly(ethylene glycol) group(s), characterized in that said IGF-1 variant has been mutated at positions 27, 37, 65 and/or 68 of the wild-type IGF-1 amino acid sequence. However, each mutation introduced into a protein in order to minimize random pegylation, at the same time increases the risk of immunogenicity. WO 2008025528 discloses the preparation of recombinant human IGF-1 fusion proteins, wherein said fusion proteins comprise amino acid substitutions at positions lysine 27, 65 and/or 68. The process described in WO2008025528 allows the preparation of recombinant human IGF-I muteins which do not bear N-terminal PEGylation. The PEGylation reagent used in WO2006066891 and WO2008025528 was the N-hydroxysuccinimidyl ester of methoxypolyethyleneglycol (PEG-NHS), which leads to randomly pegylated proteins. To avoid N-terminal PEGylation and the creation of positional isomers all lysines except one were replaced by polar amino acids and a propeptide was attached to the N-terminus. In the first step the IGF-1 mutein was PEGylated and afterwards the propeptide was cleaved from the IGF-1 with IgA protease leaving the IGF-1 mutein PEGylated only at a single lysine residue. Reductive alkylation using methoxypoly-ethyleneglycol propionaldehyde (PEG-CHO) reagent is generally recognized as a site specific PEGylation method (Roberts et al, Chemistry for peptide and protein PEGylation. 2002, Advanced Drug Delivery Reviews 54 459-476). The N-terminal PEGylation was described in the Amgene related patent family (U.S. Pat. No. 7,090,835 B2, U.S. Pat. No. 6,956,027 B2, EP 0 822199B1). The reaction of reductive alkylation was performed under acidic conditions, at pH of 5.0 (EP 0822199 B1).

### Multimers of E-Peptides:

In certain pharmacological contexts, it is beneficial to increase the size of a peptide or protein drug to ensure that the drug remains on one side of the blood-brain barrier or the other. Since mature IGF molecules are relatively short peptides, even if the E-peptide remains attached, it can be beneficial to increase the size of the polypeptides of the invention. One means of doing so is to provide multimers of E-peptides at the C-terminus of the IGF precursor polypeptide, as illustrated in certain Examples described in WO 2007/146689.

Pharmaceutical Compositions

In one aspect, the present invention provides a composition, e.g. a pharmaceutical composition, containing one or a combination of the above described IGF-1 precursor polypeptides, formulated together with a pharmaceutically acceptable carrier. Pharmaceutical compositions of the invention also can be administered in combination therapy, i.e. combined with other agents. Examples of therapeutic agents that can be used in combination therapy are described in greater detail below.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Pharmaceutically acceptable carrier include any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g. by injection or infusion). Depending on the route of administration, the active compound, i.e. antibody, immunoconjugate, or bispecific molecule, may be coated in a material to protect the compound from the action of acids and other natural conditions that may inactivate the compound.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of agents enumerated above, as required, followed by sterilization microfiltration. Generally, dispersions are prepared by incorporating the active compound into a sterile vehicle that contains a basic dispersion medium and the required other agents from those enumerated above. In the case of sterile powders for the preparation of sterile injectable solutions, the methods of preparation are vacuum drying and freeze-drying (lyophilization) that yield a powder of the active agent plus any additional desired agent from a previously sterile-filtered solution thereof.

The amount of active agent which can be combined with a carrier material to produce a single dosage form will vary depending upon the subject being treated, and the particular mode of administration. The amount of active agent which can be combined with a carrier material to produce a single dosage form will generally be that amount of the composition which produces a therapeutic effect. Generally, out of one hundred percent, this amount will range from about 0.01 percent to about ninety-nine percent of active agent, from about 0.1 percent to about 70 percent, or from about 1 percent to about 30 percent of active agent in combination with a pharmaceutically acceptable carrier.

Dosage regimens are adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

A therapeutically effective amount of a polypeptide in the context of administrating the IGF-1 precursor polypeptides of the disclosure or composition comprising said IGF-1 precursor polypeptides, ranges from about 0.001 to 10 mg/kg, or 0.01 to 3 mg/kg, and more usually 0.01 to 0.3 mg/kg of the host body weight. For example dosages can be about 0.01 mg/kg body weight, can be about 0.02 mg/kg body weight, can be about 0.03 mg/kg body weight, can be about 0.04 mg/kg body weight, can be about 0.05 mg/kg body weight, can be about 0.06 mg/kg body weight, can be about 0.1 mg/kg body weight, can be about 0.3 mg/kg body weight, can be about 0.5 mg/kg body weight or about 1 mg/kg body weight. The skilled person knows to identify a suitable effective dose, which will vary depending on the route of administration (e.g. intravenously or subcutaneously). An exemplary treatment regime entails administration once per day, once every week, once every two weeks, once every three weeks, once every four weeks or once a month. Such administration may be carried out intravenously or subcutaneously. Dosage regimens for IGF-1 precursor polypeptides of the invention include 0.01 mg/kg body weight or 0.02 mg/kg body weight or 0.03 mg/kg body weight or 0.05 mg/kg body weight or 0.1 mg/kg body weight or 0.3 mg/kg body weight or 1 mg/kg body weight by intravenous administration. Dosage regimens for IGF-1 precursor polypeptides of the invention include 0.01 mg/kg body weight or 0.02 mg/kg body weight or 0.03 mg/kg body weight or 0.05 mg/kg body weight or 0.1 mg/kg body weight or 0.3 mg/kg body weight or 1 mg/kg body weight by subcutaneous administration. For example, the dosage of the intravenously administered hIGF1-Ea-mut 3 is 0.01 mg/kg. In another embodiment of the disclosure the dosage of the intravenously administered hIGF1-Ea-mut 3 is 0.02 mg/kg. In another embodiment of the disclosure the dosage of the intravenously administered hIGF1-Ea-mut 3 is 0.03 mg/kg. In another embodiment of the disclosure the dosage of the intravenously administered hIGF1-Ea-mut 3 is 0.04 mg/kg. In another embodiment of the disclosure the dosage of the intravenously administered hIGF1-Ea-mut 3 is 0.05 mg/kg. In another embodiment of the disclosure the dosage of the intravenously administered hIGF1-Ea-mut 3 is 0.06 mg/kg. In another embodiment of the disclosure the dosage of the intravenously administered hIGF1-Ea-mut 3 is 0.1 mg/kg.

In another embodiment of the disclosure the dosage of the subcutaneously administered hIGF1-Ea-mut 3 is 0.01 mg/kg. In another embodiment of the disclosure the dosage of the subcutaneously administered hIGF1-Ea-mut 3 is 0.02 mg/kg. In another embodiment of the disclosure the dosage of the subcutaneously administered hIGF1-Ea-mut 3 is 0.03 mg/kg. In another embodiment of the disclosure the dosage of the subcutaneously administered hIGF1-Ea-mut 3 is 0.04 mg/kg. In another embodiment of the disclosure the dosage of the subcutaneously administered hIGF1-Ea-mut 3 is 0.05 mg/kg. In another embodiment of the disclosure the dosage of the subcutaneously administered hIGF1-Ea-mut 3 is 0.06 mg/kg In another embodiment of the disclosure the dosage of the subcutaneously administered hIGF1-Ea-mut 3 is 0.1 mg/kg.

Alternatively, the composition can be a sustained release formulation, in which case less frequent administration is required. Dosage and frequency vary depending on the half-life of the antibody in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active agents in the pharmaceutical compositions of the present invention may be varied so as to obtain an amount of the active agent which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Administration of a therapeutically effective dose of an IGF-1 variant comprised in the compositions of the invention can result in a decrease in severity of disease symptoms, an increase in frequency and duration of disease symptom-free periods, or a prevention of impairment or disability due to the disease affliction i.e. an increase in muscle mass and strength.

Patients will receive an effective amount of the polypeptide active ingredient i.e. an amount that is sufficient to detect, treat, ameliorate, or prevent the disease or disorder in question. Therapeutic effects may also include reduction in physical symptoms. The optimum effective amount and concentration of a therapeutic protein for any particular subject will depend upon various factors, including the patient's age size health and/or gender, the nature and extent of the condition, the activity of the particular therapeutic protein, the rate of its clearance by the body, and also on any possible further therapeutic(s) administered in combination with the therapeutic protein. The effective amount delivered for a given situation can be determined by routine experimentation and is within the judgment of a clinician. Dosage can be by a single dose schedule or a multiple dose schedule.

A composition of the present invention can be administered by one or more routes of administration using one or more of a variety of methods known in the art. As will be appreciated by the skilled artisan, the route and/or mode of administration will vary depending upon the desired results. Routes of administration for the therapeutic proteins of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. In one embodiment the antibody comprising composition is administered intravenously. In another embodiment the antibody is administered subcutaneously.

Alternatively, an IGF-1 variant comprising composition of the invention can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g. Sustained and Controlled Release Drug Delivery Systems, J. R. Robinson, ed., Marcel Dekker, Inc., New York, 1978.

Therapeutic compositions can be administered with medical devices known in the art. For example, in one embodiment, a therapeutic composition of the invention can be administered with a needleless hypodermic injection device, such as the devices shown in U.S. Pat. No. 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. Many other such implants, delivery systems, and modules are known to those skilled in the art and include those made by MicroCHIPS™ (Bedford, Mass.).

In certain embodiments, the human IGF-1 variant comprising composition of the invention can be formulated to ensure proper distribution in vivo. For example, the blood-brain barrier (BBB) excludes many highly hydrophilic compounds. To ensure that the therapeutic compounds of the invention cross the BBB (if desired); they can be formulated, for example, in liposomes. For methods of manufacturing liposomes, see, e.g. U.S. Pat. Nos. 4,522,811; 5,374,548; and 5,399,331. The liposomes may comprise one or more moieties which are selectively transported into specific cells or organs, thus enhance targeted drug delivery (see, e.g. V. V. Ranade, 1989 J. Clin Pharmacol. 29:685). Exemplary targeting moieties include folate or biotin (see, e.g. U.S. Pat. No. 5,416,016); mannosides (Umezawa et al., 1988 Biochem. Biophys. Res. Commun. 153:1038); antibodies (P. G. Bloeman et al., 1995 FEBS Lett. 357:140; M. Owais et al., 1995 Antimicrob. Agents Chernother. 39:180); surfactant protein A receptor (Briscoe et al., 1995 Am. J. Physiol. 1233:134); p120 (Schreier et al., 1994 J. Biol. Chem. 269:9090); see also K. Keinanen; M. L. Laukkanen, 1994 FEBSLett. 346:123; J. J. Killion; I. J. Fidler, 1994 lmrnunomethods 4:273.

Various delivery systems are known and can be used to administer the polypeptide of the invention, e.g., encapsulation in liposomes, microparticles, microcapsules, recombinant cells capable of expressing the protein, receptor-mediated endocytosis (see, e.g., Wu and Wu, J Biol Chem 262:4429-4432, 1987), construction of a nucleic acid as part of a retroviral, adeno-associated viral, adenoviral, poxviral (e.g., avipoxviral, particularly fowlpoxviral) or other vector, etc. Methods of introduction can be enteral or parenteral and include but are not limited to intradermal, intramuscular, intraperitoneal, intravenous, subcutaneous, pulmonary, intranasal, intraocular, epidural, and oral routes. The polypeptides can be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

In another embodiment, the active agent can be delivered in a vesicle, in particular a liposome (see Langer, Science 249:1527-1533, 1990). In yet another embodiment, the active agent can be delivered in a controlled release system. In one embodiment, a pump may be used. In another embodiment, polymeric materials can be used (see Howard et al., J Neurosurg 71:105, 1989). In another embodiment where the active agent of the invention is a nucleic acid encoding a polypeptide of the invention, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., Proc. Natl. Acad. Sci. USA 88:1864-1868, 1991), etc. Alternatively, a nucleic acid can be introduced intracellularly and incorporated within host cell DNA for expression, by homologous recombination.

### Cellular Transfection and Gene Therapy:

The present invention encompasses the use of nucleic acids encoding polypeptides of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response.

In another aspect, the invention provides a method of treating a target site, i.e., a target cell or tissue, in a human or other animal including transfecting a cell with a nucleic acid encoding a polypeptide of the invention, wherein the nucleic acid includes an inducible promoter operably linked to the nucleic acid encoding the targeting fusion polypeptide. For gene therapy procedures in the treatment or prevention of human disease, see for example, Van Brunt Biotechnology 6:1149-1154, 1998.

### Patient Groups

Patients diagnosed with SBMA, and with a diagnosis confirmed by genetic testing, will be eligible for treatment with the invention.

### Combination Therapy

This treatment may be combined with any treatment aimed at the primary cause of the muscle wasting process. Such combinations may include corticosteroids, immune suppressive agents, anti-cytokine agents, anti-cancer drugs; growth factors such as erythropoeitin, G-CSF, GM-CSF, or others; drugs used for the treatment of diabetes (including insulin and oral hypoglycemic agents), anti-tuberculosis drugs, and antibiotics. Combinations may include both small molecule and biomolecule agents.

The pharmaceutical compositions of the invention may be administered as the sole active agent or in conjunction with, e.g. as an adjuvant to or in combination to, other drugs e.g. an ActRIIB antibody, an ActRIIA antibody, a soluble ActRIIB decoy mimetic, an anti-myostatin antibody, a myostatin propeptide, a myostatin decoy protein that binds ActRIIB but does not activate it, a beta 2 agonist, a Ghrelin agonist, a SARM, GH agonists/mimetics or follistatin. For example, the drug of the invention may be used in combination with an ActRIIB antibody as disclosed in WO2010125003.

The invention is further described but not limited by the following Examples.

### BRIEF DESCRIPTION OF THE DRAWINGS

FIG. 1: Study Overview—Part A.

FIG. 2: Study Overview—Part B.

### SEQUENCES

| SEQ | | | |
|---|---|---|---|
| ID | DNA/ | | |
| NO. | PROT. | Description | Sequence |
| 1 | PROT. | hIGF-1 without | GPETLCGAELVDALQFVCGDR |
| | | the leader | GFYFNKPTGYGSSSRRAPQTG |
| | | sequence | IVDECCFRSCDLRRLEMYCAP |
| | | | LKPAKSA |
| 2 | PROT. | Ea peptide | RSVRAQRHTDMPKTQKEVHLK |
| | | | NASRGSAGNKNYRM |
| 3 | PROT. | Eb peptide | RSVRAQRHTDMPKTQKYQPPS |
| | | | TNKNTKSQRRKGVVPKTHPGG |
| | | | EQKEGTEASLQIRGKKKEQRR |
| | | | EIGSRNAECRGKKGK |
| 4 | PROT. | Ec peptide | RSVRAQRHTDMPKTQKYQPPS |
| | | | TNKNTKSQRRKGSTFEERK |
| 5 | PROT. | Wild type | GPETLCGAELVDALQFVCGDR |
| | | IGF-1-Ea | GFYFNKPTGYGSSSRRAPQTG |
| | | without | IVDECCFRSCDLRRLEMYCAP |
| | | the leader | LKPAKSARSVRAQRHTDMPKT |
| | | sequence | QKEVHLKNASRGSAGNKNYRM |
| 6 | PROT. | Protein | GPTLCGAELVDALQFVCGDRG |
| | | pad of | FYFNKPTGYGSSSRAAPQTGI |
| | | hIGF1-Ea- | VDECCFRSCDLRRLEMYCAPL |
| | | mut 03 | KPAKSAVRAQRHTDMPKTQKE |
| | | | VHLKNASRGSAGNKNYRM |
| 7 | DNA | hIGF1-Ea- | ggaccgacgctctgcggggct |
| | | mut 03 | gagctggtggatgctcttcag |
| | | | ttcgtgtgtggagacaggggc |
| | | | ttttatttcaacaagcccaca |
| | | | gggtatggctccagcagtcgg |
| | | | gcggcgcctcagacaggcatc |
| | | | gtggatgagtgctgcttccgg |
| | | | agctgtgatctaaggaggctg |
| | | | gagatgtattgcgcacccctc |
| | | | aagcctgccaagtcagctgtc |
| | | | cgtgcccagcgccacaccgac |
| | | | atgcccaagacccagaaggaa |
| | | | gtacatttgaagaacgcaagt |
| | | | agagggagtgcaggaaacaag |
| | | | aactacaggatg |
| 8 | PROT. | hIGF1-Ea- | GPTLCGAELVDALQFVCGDRG |
| | | mut-variant | FYFNKPTGYGSSSQRAPQTGI |
| | | | VDECCFRSCDLRRLEMYCAPL |
| | | | KPAKSAVRAQRHTDMPKTQKE |
| | | | VHLKNASRGSAGNKNYRM |
| 9 | PROT. | hIGF1-Ea- | GPTLCGAELVDALQFVCGDRG |
| | | mut-variant | FYFNKPTGYGSSSREAPQTGI |
| | | | VDECCFRSCDLRRLEMYCAPL |
| | | | KPAKSAVRAQRHTDMPKTQKE |
| | | | VHLKNASRGSAGNKNYRM |
| 10 | PROT. | hIGF1-Ea- | GPTLCGAELVDALQFVCGDRG |
| | | mut-variant | FYFNKPTGYGSSSRAAPQTGI |
| | | | VDECCFRSCDLRRLEMYCAPL |
| | | | KPAKSAVRAQRHTDMPKTQKE |
| | | | VHLKNASRGSAGNKNYRM |
| 11 | PROT. | hIGF1-Ea- | GPTLCGAELVDALQFVCGDRG |
| | | mut-variant | FYFNKPTGYGSSSRPAPQTGI |
| | | | VDECCFRSCDLRRLEMYCAPL |
| | | | KPAKSAVRAQRHTDMPKTQKE |
| | | | VHLKNASRGSAGNKNYRM |
| 12 | PROT. | hIGF1-Ea- | GPTLCGAELVDALQFVCGDRG |
| | | mut-variant | FYFNKPTGYGSSSQQAPQTGI |
| | | | VDECCFRSCDLRRLEMYCAPL |
| | | | KPAKSAVRAQRHTDMPKTQKE |
| | | | VHLKNASRGSAGNKNYRM |
| 13 | PROT. | hIGF1-Ea- | GPTLCGAELVDALQFVCGDRG |
| | | mut-variant | FYFNKPTGYGSSSQAAPQTGI |
| | | | VDECCFRSCDLRRLEMYCAPL |
| | | | KPAKSAVRAQRHTDMPKTQKE |
| | | | VHLKNASRGSAGNKNYRM |
| 14 | PROT. | hIGF1-Ea- | GPTLCGAELVDALQFVCGDRG |
| | | mut-variant | FYFNKPTGYGSSSQRAPQTGI |
| | | | VDECCFRSCDLRRLEMYCAPL |
| | | | KPAKSAVQAQRHTDMPKTQKE |
| | | | VHLKNASRGSAGNKNYRM |
| 15 | PROT. | hIGF1-Ea- | GPTLCGAELVDALQFVCGDRG |
| | | mut-variant | FYFNKPTGYGSSSQRAPQTGI |
| | | | VDECCFRSCDLRRLEMYCAPL |
| | | | KPAKSAVQAQQHTDMPKTQKE |
| | | | VHLKNASRGSAGNKNYRM |
| 16 | PROT. | hIGF1-Ea- | GPTLCGAELVDALQFVCGDRG |
| | | mut-variant | FYFNKPTGYGSSSQRAPQTGI |
| | | | VDECCFRSCDLRRLEMYCAPL |
| | | | KPAKSAVQAQQHTDMPKTQKE |
| | | | VHLKNASRGSAGNKNYQM |
| 17 | PROT. | hIGF1-Ea- | GPTLCGAELVDALQFVCGDRG |
| | | mut-variant | FYFNKPTGYGSSSREAPQTGI |
| | | | VDECCFRSCDLRRLEMYCAPL |
| | | | KPAKSAVRAQQHTDMPKTQKE |
| | | | VHLKNASRGSAGNKNYRM |
| 18 | PROT. | hIGF1-Ea- | GPTLCGAELVDALQFVCGDRG |
| | | mut-variant | FYFNKPTGYGSSSREAPQTGI |
| | | | VDECCFRSCDLRRLEMYCAPL |
| | | | KPAKSAVQAQQHTDMPKTQKE |
| | | | VHLKNASRGSAGNKNYRM |
| 19 | PROT. | hIGF1-Ea- | GPTLCGAELVDALQFVCGDRG |
| | | mut-variant | FYFNKPTGYGSSSREAPQTGI |
| | | | VDECCFRSCDLRRLEMYCAPL |
| | | | KPAKSAVQAQQHTDMPKTQKE |
| | | | VHLKNASRGSAGNKNYQM |
| 20 | PROT. | hIGF1-Ea- | GPTLCGAELVDALQFVCGDRG |
| | | mut-variant | FYFNKPTGYGSSSRAAPQTGI |
| | | | VDECCFRSCDLRRLEMYCAPL |
| | | | KPAKSAVQAQRHTDMPKTQKE |
| | | | VHLKNASRGSAGNKNYRM |
| 21 | PROT. | hIGF1-Ea- | GPTLCGAELVDALQFVCGDRG |
| | | mut-variant | FYFNKPTGYGSSSRAAPQTGI |
| | | | VDECCFRSCDLRRLEMYCAPL |
| | | | KPAKSAVQAQQHTDMPKTQKE |
| | | | VHLKNASRGSAGNKNYRM |
| 22 | PROT. | hIGF1-Ea- | GPTLCGAELVDALQFVCGDRG |
| | | mut-variant | FYFNKPTGYGSSSRAAPQTGI |
| | | | VDECCFRSCDLRRLEMYCAPL |
| | | | KPAKSAVQAQQHTDMPKTQKE |
| | | | VHLKNASRGSAGNKNYQM |
| 23 | PROT. | hIGF1-Ea- | GPTLCGAELVDALQFVCGDRG |
| | | mut-variant | FYFNKPTGYGSSSRPAPQTGI |
| | | | VDECCFRSCDLRRLEMYCAPL |
| | | | KPAKSAVQAQRHTDMPKTQKE |
| | | | VHLKNASRGSAGNKNYRM |
| 24 | PROT. | hIGF1-Ea- | GPTLCGAELVDALQFVCGDRG |
| | | mut-variant | FYFNKPTGYGSSSRPAPQTGI |
| | | | VDECCFRSCDLRRLEMYCAPL |
| | | | KPAKSAVQAQQHTDMPKTQKE |
| | | | VHLKNASRGSAGNKNYRM |
| 25 | PROT. | hIGF1-Ea- | GPTLCGAELVDALQFVCGDRG |
| | | mut-variant | FYFNKPTGYGSSSRPAPQTGI |
| | | | VDECCFRSCDLRRLEMYCAPL |
| | | | KPAKSAVQAQQHTDMPKTQKE |
| | | | VHLKNASRGSAGNKNYQM |
| 26 | PROT. | hIGF1-Ea- | GPTLCGAELVDALQFVCGDRG |
| | | mut-variant | FYFNKPTGYGSSSQQAPQTGI |
| | | | VDECCFRSCDLRRLEMYCAPL |
| | | | KPAKSAVQAQRHTDMPKTQKE |
| | | | VHLKNASRGSAGNKNYRM |
| 27 | PROT. | hIGF1-Ea- | GPTLCGAELVDALQFVCGDRG |
| | | mut-variant | FYFNKPTGYGSSSQQAPQTGI |
| | | | VDECCFRSCDLRRLEMYCAPL |
| | | | KPAKSAVQAQQHTDMPKTQKE |
| | | | VHLKNASRGSAGNKNYRM |
| 28 | PROT. | hIGF1-Ea- | GPTLCGAELVDALQFVCGDRG |
| | | mut-variant | FYFNKPTGYGSSSQAAPQTGI |
| | | | VDECCFRSCDLRRLEMYCAPL |
| | | | KPAKSAVQAQQHTDMPKTQKE |
| | | | VHLKNASRGSAGNKNYRM |
| 29 | PROT. | hIGF1-Ea- | GPTLCGAELVDALQFVCGDRG |
| | | mut-variant | FYFNKPTGYGSSSQQAPQTGI |
| | | | VDECCFRSCDLRRLEMYCAPL |
| | | | KPAKSAVQAQQHTDMPKTQKE |
| | | | VHLKNASRGSAGNKNYQM |
| 30 | PROT. | hIGF1-Ea- | GPTLCGAELVDALQFVCGDRG |
| | | mut-variant | FYFNKPTGYGSSSQAAPQTGI |
| | | | VDECCFRSCDLRRLEMYCAPL |
| | | | KPAKSAVQAQQHTDMPKTQKE |
| | | | VHLKNASRGSAGNKNYQM |
| 31 | PROT. | hIGF1-Ea- | GPTLCGAELVDALQFVCGDRG |
| | | mut-variant | FYFNKPTGYGSSSQRAPQTGI |
| | | | VDECCFRSCDLRRLEMYCAPL |
| | | | KPAVRAQRHTDMPKTQKEVHL |
| | | | KNASRGSAGNKNYRM |
| 32 | PROT. | hIGF1-Ea- | GPTLCGAELVDALQFVCGDRG |
| | | mut-variant | FYFNKPTGYGSSSREAPQTGI |
| | | | VDECCFRSCDLRRLEMYCAPL |
| | | | KPAVRAQRHTDMPKTQKEVHL |
| | | | KNASRGSAGNKNYRM |
| 33 | PROT. | hIGF1-Ea- | GPTLCGAELVDALQFVCGDRG |
| | | mut-variant | FYFNKPTGYGSSSRAAPQTGI |
| | | | VDECCFRSCDLRRLEMYCAPL |
| | | | KPAVRAQRHTDMPKTQKEVHL |
| | | | KNASRGSAGNKNYRM |
| 34 | PROT. | hIGF1-Ea- | GPTLCGAELVDALQFVCGDRG |
| | | mut-variant | FYFNKPTGYGSSSRPAPQTGI |
| | | | VDECCFRSCDLRRLEMYCAPL |
| | | | KPAVRAQRHTDMPKTQKEVHL |
| | | | KNASRGSAGNKNYRM |
| 35 | PROT. | hIGF1-Ea- | GPTLCGAELVDALQFVCGDRG |
| | | mut-variant | FYFNKPTGYGSSSQQAPQTGI |
| | | | VDECCFRSCDLRRLEMYCAPL |
| | | | KPAQVRAQRHTDMPKTQKEVH |
| | | | LKNASRGSAGNKNYRM |
| 36 | PROT. | hIGF1-Ea- | GPTLCGAELVDALQFVCGDRG |
| | | mut-variant | FYFNKPTGYGSSSQAAPQTGI |
| | | | VDECCFRSCDLRRLEMYCAPL |
| | | | KPAVRAQRHTDMPKTQKEVHL |
| | | | KNASRGSAGNKNYRM |
| 37 | PROT. | hIGF1-Ea- | GPTLCGAELVDALQFVCGDRG |
| | | mut-variant | FYFNKPTGYGSSSQRAPQTGI |
| | | | VDECCFRSCDLRRLEMYCAPL |
| | | | KPAVQAQRHTDMPKTQKEVHL |
| | | | KNASRGSAGNKNYRM |
| 38 | PROT. | hIGF1-Ea- | GPTLCGAELVDALQFVCGDRG |
| | | mut-variant | FYFNKPTGYGSSSQRAPQTGI |
| | | | VDECCFRSCDLRRLEMYCAPL |
| | | | KPAVQAQQHTDMPKTQKEVHL |
| | | | KNASRGSAGNKNYRM |
| 39 | PROT. | hIGF1-Ea- | GPTLCGAELVDALQFVCGDRG |
| | | mut-variant | FYFNKPTGYGSSSQRAPQTGI |
| | | | VDECCFRSCDLRRLEMYCAPL |
| | | | KPAVQAQQHTDMPKTQKEVHL |
| | | | KNASRGSAGNKNYQM |
| 40 | PROT. | hIGF1-Ea- | GPTLCGAELVDALQFVCGDRG |
| | | mut-variant | FYFNKPTGYGSSSREAPQTGI |
| | | | VDECCFRSCDLRRLEMYCAPL |
| | | | KPAVQAQRHTDMPKTQKEVHL |
| | | | KNASRGSAGNKNYRM |
| 41 | PROT. | hIGF1-Ea- | GPTLCGAELVDALQFVCGDRG |
| | | mut-variant | FYFNKPTGYGSSSREAPQTGI |
| | | | VDECCFRSCDLRRLEMYCAPL |
| | | | KPAVQAQQHTDMPKTQKEVHL |
| | | | KNASRGSAGNKNYRM |
| 42 | PROT. | hIGF1-Ea- | GPTLCGAELVDALQFVCGDRG |
| | | mut-variant | FYFNKPTGYGSSSREAPQTGI |
| | | | VDECCFRSCDLRRLEMYCAPL |
| | | | KPAVQAQQHTDMPKTQKEVHL |
| | | | KNASRGSAGNKNYQM |
| 43 | PROT. | hIGF1-Ea- | GPTLCGAELVDALQFVCGDRG |
| | | mut-variant | FYFNKPTGYGSSSRAAPQTGI |
| | | | VDECCFRSCDLRRLEMYCAPL |
| | | | KPAVQAQRHTDMPKTQKEVHL |
| | | | KNASRGSAGNKNYRM |
| 44 | PROT. | hIGF1-Ea- | GPTLCGAELVDALQFVCGDRG |
| | | mut-variant | FYFNKPTGYGSSSRAAPQTGI |
| | | | VDECCFRSCDLRRLEMYCAPL |
| | | | KPAVQAQQHTDMPKTQKEVHL |
| | | | KNASRGSAGNKNYRM |
| 45 | PROT. | hIGF1-Ea- | GPTLCGAELVDALQFVCGDRG |
| | | mut-variant | FYFNKPTGYGSSSRAAPQTGI |
| | | | VDECCFRSCDLRRLEMYCAPL |
| | | | KPAVQAQQHTDMPKTQKEVHL |
| | | | KNASRGSAGNKNYQM |
| 46 | PROT. | hIGF1-Ea- | GPTLCGAELVDALQFVCGDRG |
| | | mut-variant | FYFNKPTGYGSSSRPAPQTGI |
| | | | VDECCFRSCDLRRLEMYCAPL |
| | | | KPAVQAQRHTDMPKTQKEVHL |
| | | | KNASRGSAGNKNYRM |
| 47 | PROT. | hIGF1-Ea- | GPTLCGAELVDALQFVCGDRG |
| | | mut-variant | FYFNKPTGYGSSSRPAPQTGI |
| | | | VDECCFRSCDLRRLEMYCAPL |
| | | | KPAVQAQQHTDMPKTQKEVHL |
| | | | KNASRGSAGNKNYRM |
| 48 | PROT. | hIGF1-Ea- | GPTLCGAELVDALQFVCGDRG |
| | | mut-variant | FYFNKPTGYGSSSRPAPQTGI |
| | | | VDECCFRSCDLRRLEMYCAPL |
| | | | KPAVQAQQHTDMPKTQKEVHL |
| | | | KNASRGSAGNKNYQM |
| 49 | PROT. | hIGF1-Ea- | GPTLCGAELVDALQFVCGDRG |
| | | mut-variant | FYFNKPTGYGSSSQQAPQTGI |
| | | | VDECCFRSCDLRRLEMYCAPL |
| | | | KPAVQAQRHTDMPKTQKEVHL |
| | | | KNASRGSAGNKNYRM |
| 50 | PROT. | hIGF1-Ea- | GPTLCGAELVDALQFVCGDRG |
| | | mut-variant | FYFNKPTGYGSSSQQAPQTGI |
| | | | VDECCFRSCDLRRLEMYCAPL |
| | | | KPAVQAQQHTDMPKTQKEVHL |
| | | | KNASRGSAGNKNYRM |
| 51 | PROT. | hIGF1-Ea- | GPTLCGAELVDALQFVCGDRG |
| | | mut-variant | FYFNKPTGYGSSSQAAPQTGI |
| | | | VDECCFRSCDLRRLEMYCAPL |
| | | | KPAVQAQQHTDMPKTQKEVHL |
| | | | KNASRGSAGNKNYRM |
| 52 | PROT. | hIGF1-Ea- | GPTLCGAELVDALQFVCGDRG |
| | | mut-variant | FYFNKPTGYGSSSQQAPQTGI |
| | | | VDECCFRSCDLRRLEMYCAPL |
| | | | KPAVQAQQHTDMPKTQKEVHL |
| | | | KNASRGSAGNKNYQM |
| 53 | PROT. | hIGF1-Ea- | GPTLCGAELVDALQFVCGDRG |
| | | mut-variant | FYFNKPTGYGSSSQAAPQTGI |
| | | | VDECCFRSCDLRRLEMYCAPL |
| | | | KPAVQAQQHTDMPKTQKEVHL |
| | | | KNASRGSAGNKNYQM |

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. Unless defined otherwise, all technical and scientific terms used herein have the same meaning as commonly understood by one of ordinary skill in the art to which this disclosure belongs. The following examples are presented in order to more fully illustrate the preferred embodiments of the disclosure. These examples should in no way be construed as limiting the scope of the disclosed patient matter, as defined by the appended claims.

### Examples

### A. General Description

Active Compound: The underlying hypothesis for the following study is that IGF-1/Akt-mediated inhibition of mutant AR toxicity may be an effective strategy to treat SBMA in vivo. It is hypothesize that hIGF1-Ea-mut 3 will specifically reduce mutant AR toxicity directly attenuating muscle degeneration and improving function in patients with SBMA.

hIGF1-Ea-mut 3 is a human IGF-1 (hIGF-1) mimetic whose sequence has been modified to increase its efficacy, by reducing proteolytic degradation, decreasing binding to inhibitory IGFBP5 and by adding a linear polyethylene glycol (PEG) chain.

### 1. Study Purpose/Objectives and Investigational Plan

The purpose of this study is to evaluate the safety, tolerability and preliminary efficacy of the IGF-1 mimetic hIGF1-Ea-mut 3 in patients with SBMA who have reduced levels of IGF-1. The study is designed as a two-part, double-blind, placebo-controlled study.

The purpose of Part A of this study is to confirm the safety and tolerability of selected doses of hIGF1-Ea-mut 3 in patients with SBMA, and to preliminarily investigate its pharmacodynamic effects on the target tissue.

Following successful demonstration of safety and tolerability of hIGF1-Ea-mut 3 in patients with SBMA in Part A, the therapeutic efficacy of a single dose (to be determined in Part A) of hIGF1-Ea-mut 3 administered weekly will be investigated in Part B.

### Investigational Plan

This is a double-blind, randomized, placebo-controlled, non-confirmatory study in approximately 38 patients with SBMA.

The study is conducted in two parts, Part A and Part B. Part A must be completed before Part B can start. Patients dosed in Part A of the study may participate in Part B after a “wash-out” period of at least 60 days. Patients will be required to consent to Part B and screening and baseline visit assessments will be repeated in order for them to qualify for the second portion of the study.

There are three planned interim analyses. During the interim analyses, safety, pharmacokinetic, and pharmacodynamic data will be reviewed. This review will include adverse events, safety labs, pharmacokinetic data, IGF-1 like activity, IGF-1 antibodies, and IGFBPs. The interim analyses will be conducted by an internal Novartis team. In addition, during each interim analysis, an independent Data Monitoring Committee (DMC) will conduct a separate review of all safety related data. Interim analyses are scheduled throughout the course of the trial
1. After the first two open label patients have completed the trial, progression to the randomized portion of Part A will proceed following the first interim analysis.
2. At the completion of Part A, in addition to a safety and a pharmacodynamic data review, PK analyses will confirm the dose and interval of therapy to utilize in Part B. The maximum dose judged to be well-tolerated and with confirmed PD effect in Part A will be utilized in Part B.
3. An interim analysis will be performed after 12 patients complete Part B of the study.

### 2. Study Design

### 2.1 Part A

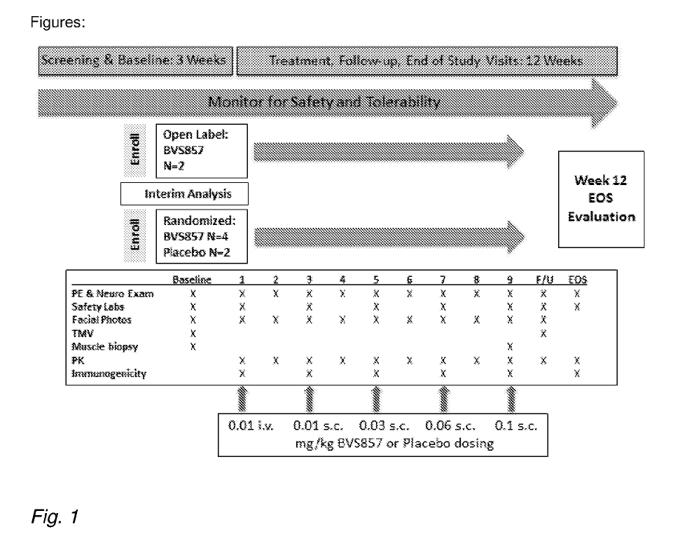

This part of the study will consist of a screening period, a baseline period, a treatment period with 5 doses, follow-up visits and a study completion evaluation. An overview of the study design for Part A is presented in FIG. 1.

Safety assessments throughout Part A include physical examinations, ECGs, vital signs, standard clinical laboratory evaluations (hematology, blood chemistry, urinalysis, coagulation panel), glucose monitoring, adverse event and serious adverse event monitoring, facial photos, fundus exam, visual acuity, and immunogenicity. Pharmacodynamic assessments include IGF-1, total IGF-1 like activity, cellular biomarkers, IGF binding proteins, soluble protein markers, and muscle biopsy.

### 2.1.1 Screening

Patients are required to attend the investigator center for the screening visit where suitability for the study will be assessed by the Investigator. Patients who meet the eligibility criteria at screening will be asked to attend for baseline evaluations.

### 2.1.2 Baseline

Following completion of a successful screening visit, patients will be asked to return to the clinic for baseline evaluations. Patients who pass the required baseline assessments will be eligible to proceed to the treatment period.

All baseline results required for inclusion into the trial should be available and reviewed by the Investigator prior to a patient being randomized and progressing to the treatment period.

### 2.1.3 Treatment

Following successful completion of baseline assessments, the first two patients will receive open-label active drug (hIGF1-Ea-mut 3). Dose levels will be increased as shown in the table below. After these two patients complete treatment and follow-up, the first interim analysis will occur. Following a satisfactory review from the interim analysis, the remaining six patients in Part A will be randomized to receive five doses of either hIGF1-Ea-mut 3 or placebo at a ratio of 2:1. Pharmacokinetic assessments throughout this period will allow for the assessment of the bioavailability of hIGF1-Ea-mut 3 in SBMA patients.

The dosing schedule for Part A is shown in the table below:

TABLE (a)
Dosing in Part A

| | | | Dose of hIGF1-Ea- | |
|---|---|---|---|---|
| | Approx. No. | | mut 3 (mg/kg) or | Route of |
| Cohort | of patients | Study Week | placebo | admin. |
| 1 | 2 | 1 | 0.01 | i.v. |
| | (hIGF1-Ea- | 3 | 0.01 | s.c. |
| | mut 3, open- | 5 | 0.03 | s.c. |
| | label) | 7 | 0.06 | s.c. |
| | | 9 | 0.10 | s.c. |
| 2 | 6 | 1 | 0.01 | i.v. |
| | (4 hIGF1-Ea- | 3 | 0.01 | s.c. |
| | mut 3 and 2 | 5 | 0.03 | s.c. |
| | placebo, | 7 | 0.06 | s.c. |
| | double-blind) | 9 | 0.10 | s.c. |

On dosing visits, patients will be admitted to the study site approximately 2 hours prior to each dose and will remain domiciled until completing the assessments at 24 hours post-dose. Prior to and following dosing, safety, pharmacokinetic and pharmacodynamic assessments will be conducted. Patients will return to the study site for 48 hour and weekly post-dose assessments.

### 2.1.4 End of Study (EOS)

Patients will return to the clinic for an outpatient visit following their last administration of hIGF1-Ea-mut 3 or placebo for pharmacokinetic, and study completion evaluations.

### 2.2. Part B

An interim analysis will be conducted at the end of Part A. Dosing in Part B will be determined by PK, PD and safety assessments in Part A. Thirty patients will be randomly assigned to receive the dose of hIGF1-Ea-mut 3 determined to be well-tolerated in Part A (up to 0.1 mg/kg) or placebo [2:1 ratio] administered subcutaneously. After the first 12 randomized patients have completed Part B, an interim analysis will be conducted to review PK, PD and safety data. This part of the study will consist of a screening period, a single baseline period, twelve treatment visits and a study completion evaluation.

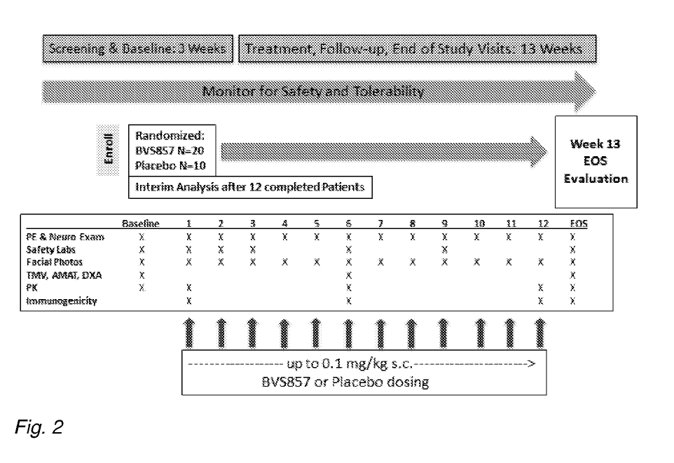

An overview of the study design for Part B is presented in FIG. 2.

Safety assessments throughout Part B include physical examinations, ECGs, vital signs, standard clinical laboratory evaluations (hematology, blood chemistry, urinalysis, coagulation panel), glucose monitoring, adverse event and serious adverse event monitoring, facial photos, fundus exam, visual acuity, and immunogenicity. Pharmacodynamic assessments include IGF-1, total IGF-1 like activity, cellular biomarkers, IGF binding proteins, and soluble protein markers. Efficacy measures include TMV by MRI, AMAT, LBM by DXA and measures of muscle strength and function.

### 2.2.1 Screening

Patients are required to attend the investigator center for the screening visit where suitability for the study will be assessed by the Investigator. Patients who meet the eligibility criteria at screening will be asked to attend for baseline evaluations.

### 2.2.2 Baseline

Following completion of a successful screening visit, patients will be asked to return to the clinic for baseline evaluations. Patients who pass the required baseline assessments will be eligible to proceed to the treatment period. As noted, patients who have completed Part A will be eligible for Part B, but will be required to complete all associated visit assessments for Part B.

All baseline results required for inclusion in the trial should be available and reviewed by the Investigator prior to progressing to the treatment period.

### 2.2.3 Treatment

Following successful completion of baseline assessments patients will be randomized to receive twelve doses of either hIGF1-Ea-mut 3 or placebo at a ratio of 2:1.

The dosing schedule for Part B is presented in the table below.

TABLE (b)
Dosing in Part B

| | | | Dose of hIGF1- | |
|---|---|---|---|---|
| | Approx. No. | Dosing | Ea-mut 3 or | Route of |
| Cohort | of patients | frequency | placebo (mg/kg) | admin. |
| 3 | 30 | Once weekly* | ≦0.1 | s.c. |
| | (20 hIGF1- | for 12 weeks | | |
| | Ea-mut 3, | | | |
| | 10 placebo) | | | |

*Dosing frequency may be adjusted depending on the pharmacokinetic, pharmacodynamics and safety results of an interim analysis from Part A.

On dosing visits, patients will be admitted to the study site approximately 2 hours prior to each dose and will be discharged following completion of the 4 hour assessments. All visits are outpatient visits.

### 2.2.4 End of Study (EOS)

Patients will undergo Study Completion evaluations and will be discharged from the study one week after the final dose of study drug.

### 3. Rationale for Study Design

In Part A, 5 doses of hIGF1-Ea-mut 3 or placebo (0.01 i.v., 0.01 s.c., 0.03 s.c., 0.06 s.c., and 0.1 s.c. mg/kg) will be administered every 2 weeks and the patients will be followed for 3 weeks after the last dose. As an additional safety measure, the first two patients will receive open-label hIGF1-Ea-mut 3 and will be followed until study completion. Safety assessments include physical/neurological examinations, ECGs, vital signs, standard clinical laboratory evaluations (hematology, blood chemistry, urinalysis, coagulation panel), glucose monitoring, adverse event and serious adverse event monitoring, IGF-1, total IGF-1 like activity, facial photos, fundus exam, visual acuity and immunogenicity. The first dose will be administered i.v. and subsequent doses s.c. in order to describe the pharmacokinetics and to assess the bioavailability of hIGF1-Ea-mut 3 in SBMA patients. The escalating s.c. doses will allow for a reduced peak to trough ratio as compared to the i.v. route, thus minimizing the potential risk associated with higher Cmax. Thigh muscle volume (TMV) and muscle biopsy (part A only) will be performed at baseline and post-treatment for PD measures. For the former (TMV), the observation of an effect on the target tissue will provide further support for the potential success of Part B. PD measures in muscle tissue obtained at biopsy will provide additional data regarding whether hIGF1-Ea-mut 3 at the tested doses has measurable effects in muscle.

A parallel-arm design was chosen since it is desirable to maximize duration of therapy in a chronic disease with a direct comparison to untreated patients. A 2:1 active: placebo ratio was chosen to enhance recruitment of eligible patients while maintaining statistical significance. The duration of the trial (12 weeks) is based on the availability of preclinical toxicology for 13 weeks of therapy, and as noted, sustained treatment is likely necessary to optimize likelihood of detecting a therapeutic response.

Part B is designed to determine the preliminary efficacy of hIGF1-Ea-mut 3 at a dose regimen determined to be well-tolerated in Part A (up to 0.1 mg/kg) administered weekly for 12 weeks in patients with SBMA. In addition to safety, the primary efficacy measure (TMV) was selected based on likelihood of observing a treatment effect given the mechanism of action of hIGF1-Ea-mut 3 on muscle mass/volume. Secondary and exploratory measures of muscle strength and function will determine if changes in muscle volume are functionally relevant in SBMA patients.

### 4. Rationale for Dose/Regimen, Duration of Treatment

A dose regimen of 0.01 i.v., 0.01 s.c., 0.03 i.v., 0.03 s.c., 0.06 s.c. and 0.1 s.c. mg/kg was chosen to gain PK and PD information rapidly in small number of patients to choose the appropriate dose regimen in Part B.

In Part A, total IGF-1-like activity will be reviewed after the first two open-label patients to ensure that the ceiling exposure value is not exceeded. Similarly, after completion of Part A. these data will be reviewed and assessed to be acceptable prior to moving on to Part B. In Part B, total IGF-1 like activity will be reviewed at an interim analysis, after at least 12 patients have completed the study. These additional precautions should provide adequate safety in SBMA patients, given the inclusion requirement of reduced IGF-1 levels/activity, hence avoiding the adverse findings associated with supra-physiologic IGF-1 levels. Enrolled patients must have low baseline serum levels of IGF-1—less than 170 ng/mL, which is more than one standard deviation (SD) below the mean of healthy control males aged 40-60 years (Colao A, Di Somma C, Cascella T, et al (2008) Relationships between serum IGF-1 levels, blood pressure and glucose tolerance; an observational exploratory study in 404 subjects. Eur J Endocrinol; 159:389-97).

The dose and dosing interval for Part A has been selected based on the observed half-life of hIGF1-Ea-mut 3. The dosing interval may be modified based on the results of Part A, which will provide the first opportunity to dose patients with SBMA both i.v. and s.c.

### 5. Rationale for Choice of Comparator

Placebo control is proposed since there is no known therapy for SBMA, and because the assessment of muscle strength and function requires the use of tests measuring physical performance (such as timed up-and-go, timed walk, quantitative muscle testing) which are affected by patient and observer participation and motivation, potentially leading to bias. There are currently no effective treatments for SBMA available to replace the use of placebo as a comparator.

### 6. Purpose and Timing of Interim Analyses/Design Adaptations

During the interim analyses, safety, pharmacokinetic, and pharmacodynamic data will be reviewed. This review will include adverse events, safety labs, pharmacokinetic data, IGF-1 like activity, IGF-1 antibodies, and IGFBPs. The study stopping criteria will be used as a guide for the analyses. The interim analyses will be conducted by an unblinded review committee (a sub team of the Clinical Trial Team consisting of the Translational Medicine Expert, Statistician, Clinical Trial Leader, Biomarker Expert and PK Expert).

In addition, during each interim analysis, an independent Data Monitoring Committee (DMC) will conduct a separate review of all safety related data.

Additional interim analyses may be conducted to support decision making concerning the current clinical study, clinical development projects in general or in case of any safety concerns.

### 6.1 Interim Analysis 1: Open Label Phase (Initial Two Patients of Part A)

As a safety measure, after the first two patients have completed dosing and a 3 week follow-up period, an interim analysis will be performed. Progression to the randomized portion of Part A will proceed once safety/tolerability is confirmed and the PK profile is satisfactory.

6.2 Interim Analysis 2: Transition from Part A to Part B

At the completion of Part A, safety, pharmacokinetic, and pharmacodynamics data will be reviewed to confirm the dose and interval of therapy to utilize in Part B. The maximum dose judged to be well-tolerated in Part A will be utilized in Part B.

### 6.3 Interim Analysis 3: Part B

To ensure the proper dose and interval was chosen for Part B, an interim analysis will be performed after at least 12 patients complete Part B.

A preliminary review of efficacy data may also be performed during this interim analysis, the review would be conducted in an unblinded fashion by the Translational Medicine Expert, the Statistician, the Clinical Trial Leader and the PK expert and assessed as a preliminary efficacy evaluation. The purpose of this interim analysis is to support early decision making concerning the current clinical study as well as clinical development projects in general.

### 7. Population

The study population will be comprised of male SBMA patients. A total of approximately 38 patients will be enrolled to participate in the study and randomized.

Subject selection is to be established by checking through all inclusion/exclusion criteria at screening and baseline visits, as specified below. Deviation from any entry criterion excludes a subject from enrollment into the study.

### 8. Inclusion Criteria:

Subjects eligible for inclusion in this study have to fulfill all of the following criteria:
1. Written informed consent must be obtained before any assessment is performed.
2. Males aged 18 or greater with a confirmed genetic diagnosis of SBMA and symptomatic muscle weakness.
3. Serum IGF-1≦170 ng/mL at screening.
4. Able to complete 2 minute timed walk with or without the aid of an assisted device at screening and baseline.
5. Able to communicate well with the investigator, to understand and comply with the requirements of the study.

### 9. Exclusion Criteria

Patients fulfilling any of the following criteria are not eligible for inclusion in this study
1. Use of other investigational drugs at the time of enrollment, or within 5 half-lives of enrollment, or until the expected PD effect has returned to baseline, whichever is longer, or longer if required by local regulations.
2. History of hypersensitivity to the study drug or to drugs of similar chemical classes.
3. Medically treated diabetes mellitus, or known history of hypoglycemia.
4. History of Bell's palsy, raised intracranial pressure, papilledema, pseudotumor cerebri, or retinopathy.
5. Severe facial weakness as documented by a score of 1 or 2 on items A or B on the Bulbar Rating Scale at screening or baseline.
6. Use of drugs known to affect muscle metabolism within the previous 3 months, including systemic corticosteroids (>10 mg/day prednisone or equivalent), androgens or androgen-reducing agents, or systemic beta agonists or beta blockers, or relevant herbal or nutraceutical products.
7. History of cancer, other than non-melanomatous skin cancer which has been resected.
8. Known history of clinically significant cardio-vascular disease [including uncontrolled hypertension, ischemic heart disease (e.g., myocardial infarction, angina, abnormal coronary arteriography or cardiac stress testing/imaging), supraventricular or ventricular arrhythmias, heart failure or LV dysfunction], or clinically significant cerebro-vascular disease (stroke or transient ischemic attacks).
9. An abnormal ECG at screening or baseline visit which is judged to be clinically relevant and represent an unacceptable risk for study participation by the site investigator.
10. Any surgical or medical condition which may jeopardize the patient in case of participation in the study. The Investigator should make this determination in consideration of the patient's medical history and/or clinical or laboratory evidence of any of the following:
   Inflammatory bowel disease, ulcers, gastrointestinal or rectal bleeding;
   Liver disease or liver injury as indicated by abnormal liver function tests such as SGOT (AST), SGPT (ALT), γ-GT, alkaline phosphatase (ALP), or serum bilirubin in the presence of normal serum creatine kinase (CK).

The Investigator should be guided by the following criteria:
Any single parameter may not exceed 3× upper limit of normal (ULN). A single parameter elevated up to and including 3×ULN should be re-checked once more as soon as possible, and in all cases, at least prior to enrollment/randomization, to rule out lab error. For abnormal liver function tests, ALT and AST up to 5× upper limit of normal are acceptable in the presence of serum CK>1000 IU/L.
If serum CK>1000 IU/L, ALT and AST elevation 5×ULN is acceptable as long as other liver tests are normal.
If the total bilirubin concentration is increased above 1.5×ULN, total bilirubin should be differentiated into the direct and indirect reacting bilirubin. In any case, serum bilirubin should not exceed the value of 1.6 mg/dL (27 μmol/L).
11. History of immunodeficiency diseases, including a positive HIV (ELISA and Western blot) test result at screening.
12. A positive Hepatitis B surface antigen (HBsAg) or Hepatitis C test result at screening.
13. Patients with known claustrophobia, presence of pacemaker and/or ferromagnetic material in their body that would preclude MRI assessments.
14. Patients with known bleeding disorders, or who are under treatment with anti-coagulants.
15. History of drug or alcohol abuse within the 12 months prior to dosing, or evidence of such abuse as indicated by the laboratory assays conducted during screening.

No additional exclusions may be applied by the investigator, in order to ensure that the study population will be representative of all eligible subjects.

### 10. Treatment

### 10.1 Protocol Requested Treatment

The subject's weight assessed at baseline will be used for the calculation of drug dose.

hIGF1-Ea-mut 3 10 mg will be provided as lyophilisate in vial. This has to be reconstituted with water for injection and administered either via s.c. or i.v. A placebo will also be provided. i.v. infusions will take place over a minimum of one hour. A one hour saline flush will follow the infusion.

### 10.2 Treatment Arms

### 10.2.1 Part A

The first two patients will receive open-label hIGF1-Ea-mut 3 as represented in Sequence 1 in the table below. The next six patients will be randomized to one of two treatment sequences in a ratio of 2:1.

TABLE (a)
Treatment sequences

| Sequence | Day 1 | Day 15 | Day 29 | Day 43 | Day 57 |
|---|---|---|---|---|---|
| 1 | A | B | C | D | E |
| 2 | F | G | G | G | G |

Study treatments are defined as:
A: single dose of 0.01 mg/kg hIGF1-Ea-mut 3 i.v.
B: single dose of 0.01 mg/kg hIGF1-Ea-mut 3 s.c.
C: single dose of 0.03 mg/kg hIGF1-Ea-mut 3 s.c.
D: single dose of 0.06 mg/kg hIGF1-Ea-mut 3 s.c.
E: single dose of 0.10 mg/kg hIGF1-Ea-mut 3 s.c.
F: single dose of placebo to hIGF1-Ea-mut 3 i.v.
G: single dose of placebo to hIGF1-Ea-mut 3 s.c.

### 10.2.2 Part B

Patients will be randomized in a ratio of 2:1 and assigned to receive weekly doses of either 0.10 mg/kg hIGF1-Ea-mut 3 s.c. or placebo to hIGF1-Ea-mut 3 s.c. The dose and interval may be adjusted based on the data reviewed during the Part A interim analyses.

### 11. Vital Assessment

### 11.1 Efficacy and Pharmacodynamic Assessments

### 11.1.1 Thigh Muscle Volume (TMV) by MRI

Thigh muscle volume is a primary outcome of this study and will be assessed by MRI. As adipose tissue surrounding and infiltrating muscle can be related to the metabolic and functional abnormalities of the skeletal muscle in muscle wasting, the MRI pulse sequence used will also allow for lipid quantification in the thigh muscle region (i.e. subcutaneous fat-SC and intermuscular adipose tissue-IMAT).

### Data Collection and Processing

Subjects will be imaged using a similar scanner in all sites (1.5T) and a Q-body coil. Caution will be taken to ensure minimal patient motion during scanning (e.g. by placing folded pads/sheets under the legs) and same positioning used for all subsequent scans.

After a rapid survey scan, thigh images will be acquired using a 2D multislice pulse sequence to cover the entire thigh (knee-to-hip). The total sequence time will be fast enough to minimize patient discomfort. A proton-density fast-spin echo (FSE) MRI pulse sequence was considered as a favorable approach since it allows for image acquisition throughout the upper leg in a relatively short time without loss of image quality and generates good contrast between muscle and surrounding fat tissue.

### 11.1.2 Bulbar Rating Scale

The Bulbar Rating Scale (BRS) includes eight domains each rated on a 1-4 scale, abnormal to normal (Fernandez-Rhodes L E, Kokkinis A D, White M J et al (2011) Efficacy and safety of dutaseride in patients with spinal and bulbar muscular atrophy: a randomized placebo-controlled trial. Lancet Neurol; 10:140-7). The domains consist of the following muscle groups/functions: orbicularis oculi, orbicularis oris, jaw opening, jaw closure, tongue protrusion, tongue deviation, soft palate elevation, and posterior pharyngeal wall constriction. Each domain is assessed in terms of strength/function by bedside examination/observation by the investigator.

### Data Collection and Processing

Each domain will be rated on a 1-4 scale by the investigator; the respective scores in each domain will be added to obtain the BRS score (8-32). The BRS along with instructions will be provided in a separate manual. Results of the BRS score will be captured in the CRFs.

Descriptive statistics such as mean, standard deviation (SD), and standard error (SE) will be calculated to characterize the BRS results.

### Efficacy Parameter

Change from baseline will be assessed in hIGF1-Ea-mut 3-treated patients compared to placebo.

### 11.1.3 Adult Myopathy Assessment Tool (AMAT)

The Adult Myopathy Assessment Tool (AMAT) (Fernandez-Rhodes L E, Kokkinis A D, White M J et al (2011) Efficacy and safety of dutaseride in patients with spinal and bulbar muscular atrophy: a randomized placebo-controlled trial. Lancet Neurol; 10:140-7) rates physical function and muscle endurance, with higher scores indicating better performance; it includes 7 timed functional tasks and 6 endurance tasks (0=worst, 45=best). It evaluates muscles (axial and proximal limb muscles) and functions (shoulder/hip girdle, axial weakness) particularly affected by SBMA. Timed or repeated measurements for proximal and axial muscle groups are employed. The AMAT must be performed by physicians or evaluators experienced in the management of patients with SBMA.

### Data Collection and Processing

The AMAT consists of 13 functional/endurance tasks: sustained head elevation, supine to prone, modified push-up, repeated modified push-ups, sit-up, supine to sit, arm raise, sustained arm raise, sit to stand, sustained hip flexion, sustained knee extension, repeated heel raises, and step up. Each task is scored by the investigator (scale 0 [worse/weakest] to 3 or 4 [strongest]. The assessment tool along with instructions will be provided in a separate manual. Results of the AMAT will be captured in the CRFs.

### 11.1.4 Total Lean Body Mass (LBM) by Dual-Energy X-Ray Absorptiometry (DXA) Scan

DXA will be used during the study to monitor for changes in total lean body mass (LBM), which in large part reflects skeletal muscle mass. DXA instruments use an x-ray source that generates or is split into two energies to measure bone mineral mass and soft tissue from which fat and fat-free mass are estimated. The exam is quick (1-2 min), precise (0.5-1%) and non-invasive. DXA scanners have the precision required to detect changes in muscle mass as small as 5%.

Radiation exposure from DXA scans is minimal. The National Council of Radiation Protection and Measurements (NCRP) has recommended the annual effective dose limit for infrequent exposure of the general is 5,000 μSv and that an annual effective dose of 10 μSv be considered a Negligible Individual Dose. The effective dose of a dual-energy x-ray absorptiometry whole body scan on an adult is 2.1 μSv.

Studies have shown that quality assurance is an important issue in the use of DXA scans to determine body composition. DXA instrument manufacturer and model should remain consistent and their calibration should be monitored throughout the study. Use of a standardized scan acquisition protocol and appropriate and unchanging scan acquisition and analysis software is essential to achieve consist results. Likewise, because of variability in interpretation of the scans, it is important to utilize centralized scan analysis by experienced staff.

### 11.1.5 Quantitative Muscle Testing (QMT)

Quantitative muscle testing (QMT; also called Maximum Voluntary Isometric Contraction Test (MVICT)) will be performed using the QMA system (Computer Source, Atlanta, Ga.) or the Biodex system (Biodex Medical Systems). These systems use an adjustable cuff to attach the patient's arm or leg to an inelastic strap that is connected to a force transducer with a load of 0.5 to 1,000 Newtons.

### 11.1.6 Timed Up-and-go (TUG) Test

The test assesses a person's ability to rise from the seated position, walk 3 meters, turn 1800, walk back to the chair, turn around again and sit down. A walking aid can be used to perform the test if necessary. A tape measure, stopwatch, and a standard height chair are required. (Rutkove S B, Parker R A, Nardin R A, et al (2002) A pilot randomized trial of oxandrolone in inclusion body myositis. Neurology; 58:1081-7)

### 11.17 2 Minute or 6 Minute Timed Walk

In order to qualify for Part A of the trial, patients will be required to perform a 2 minute timed walk at screening and baseline. The 6 minute walk test is being modified to 2 minutes for inclusion into the trial.

In order to qualify for the trial for Part B, patients will be required to perform a 2 minute timed walk at screening and baseline, but will be requested to perform an entire 6 minute walk test if they are able to do so.

The 6 minute walk test assesses the distance a patient can walk in 6 minutes (Rutkove S B, Parker R A, Nardin R A, et al (2002) A pilot randomized trial of oxandrolone in inclusion body myositis. Neurology; 58:1081-7). This is a widely utilized clinical test that assesses the functional capacity of gait. The distance traveled during 6 min, i.e. the 6-min walk distance is a parameter that evaluates the global and integrated responses of all the systems involved in walking, including the neuromuscular, pulmonary and cardiovascular systems. The validity of this test has been verified in various neuromuscular disorders, including spinal and bulbar muscular atrophy.

After the patient qualifies for the trial in Part B, if they are not able to walk for 6 minutes, a 2 minute walking test (2MWT) will be conducted instead.

### LIST OF ABBREVIATIONS

AE adverse event
AESI adverse event of special interest
ALT alanine aminotransferase
ALP alkaline phosphatase
AMAT adult myopathy assessment tool
ANCOVA analysis of covariance
aPTT activated partial thromboplastin time
AR androgen receptor
AST aspartate aminotransferase
BMI Body Mass Index
BRS bulbar rating scale
CFR Code of Federal Regulation
CK creatinine kinase
CN cranial nerve
CRF Case Report Form
CRO Contract Research Organization
CV coefficient of variation
DMC Data Monitoring Committee
DXA Dual-energy X-ray absorptiometry
EC Ethics committee
ECG Electrocardiogram
ELISA Enzyme-linked immunosorbent assay
EOS End of Study
FDA Food and Drug Administration
GCP Good Clinical Practice
γ-GT Gamma-glutamyl transferase
HAQ Health Assessment Questionnaire
HAQ-DI Health Assessment Questionnaire Disability Index
hIGF-1 human IGF-1
hIGF1-Ea-mut 3 human IGF-1 precursor polypeptide as shown in SEQ ID NO: 6 comprising a linear poly(ethylene glycol) moiety having an overall molecular weight of about 30 kDa covalently attached to the N-terminus of said protein.
HIV human immunodeficiency virus
ICH International Conference on Harmonization of Technical Requirements for Registration of Pharmaceuticals for Human Use
IEC Independent Ethics Committee
IGF-1 Insulin-like growth factor-1
IGFBPs IGF Binding Proteins
IGFBP3 IGF Binding Protein 3
IGFBP5 IGF Binding Protein 5
i.v. intravenous
IR insulin resistant
IRB Institutional Review Board
LBM lean body mass
LLOQ lower limit of quantification
MRI Magnetic Resonance Imaging
MTD maximum tolerated dose
PD pharmacodynamic(s)
PEG polyethylene glycol
PG pharmacogenetics
PK pharmacokinetic(s)
QMT quantitative muscle testing
REB Research Ethics Board
SAE serious adverse event
SBMA spinal and bulbar muscular atrophy
s.c. subcutaneous
SGOT serum glutamic oxaloacetic transaminase
SGPT serum glutamic pyruvic transaminase
SD standard deviation
TBL total bilirubin
TMV thigh muscle volume
TUG timed up and go
ULN upper limit of normal
ULOQ upper limit of quantification

1. A method for treating Spinal and Bulbar Muscular Atrophy (SBMA) in a patient suffering from SBMA, the method comprising administering to said patient a therapeutically effective amount of an IGF-1 mimetic, wherein the IGF-1 mimetic is a polypeptide comprising an IGF-1 precursor protein comprising the E-peptide.

2. A method for preventing, ameliorating or reversing symptoms associated with SBMA in a patient suffering from SBMA, the method comprising administering to said patient a therapeutically effective amount of an IGF-1 mimetic, wherein the IGF-1 mimetic is a polypeptide comprising an IGF-1 precursor protein comprising the E-peptide.

3. The method according to claim 1 which results in reducing or preventing degeneration of motor neurons or in preventing or reversing skeletal muscle weakness and/or atrophy, in a patient suffering from SBMA.

4. A method for reducing or preventing degeneration of motor neurons or for preventing or reversing skeletal muscle weakness and/or atrophy, in a patient suffering from SBMA, the method comprising administering to said patient a therapeutically effective amount of an IGF-1 mimetic, wherein the IGF-1 mimetic is a polypeptide comprising an IGF-1 precursor protein comprising the E-peptide.

5. The method according to claim 1 which results in reducing mutant androgen receptor (AR) aggregation in skeletal muscle to reduce mutant AR toxicity.

6. The method according to claim 1, wherein the precursor protein is a human IGF-1 precursor protein.

7. The method according to claim 1, wherein the precursor protein is modified such that the cleavage of the E-peptide from IGF-1 by a protease is reduced.

8. The method according to claim 1, wherein the precursor protein comprises the Ea, Eb or Ec peptide.

9. The method according to claim 8, wherein the precursor protein comprises the Ea peptide.

10. The method according to claim 9, wherein one or more of amino acid residues E3, R71 or S72 of the precursor protein are deleted, wherein the numbering of the amino acids corresponds to SEQ ID NO: 5.

11. The method according to claim 9, wherein the arginine at position 37 of the precursor protein is mutated to an alanine (R37A).

12. The method according to claim 9, wherein the precursor protein comprises the following modification: ΔE3; R37A; ΔR71, ΔS72, wherein the numbering of the amino acids corresponds to SEQ ID NO: 5.

13. The method according to claim 1, wherein the precursor protein comprises the amino acid sequence as shown in SEQ ID NO: 6.

14. The method according to claim 1, wherein the IGF-1 mimetic further comprises a poly(ethylene glycol) moiety covalently attached to a side-chain of the precursor protein.

15. The method according to claim 14, wherein the pegylated precursor protein comprises the amino acid sequence as shown in SEQ ID NO: 6.

16. (canceled)

17. (canceled)

18. (canceled)

19. (canceled)

20. The method according to claim 1, wherein the IGF-1 mimetic is to be administered at a dose of 0.001-1 mg/kg body weight.

21. The method according to claim 20, wherein the IGF-1 mimetic is to be administered at a dose of about 0.01, about 0.03, about 0.06, about 0.1, about 0.3, about 0.5, about 1 mg/kg body weight.

22. The method according to claim 1, wherein the IGF-1 mimetic is to be administered as a single intravenous or subcutaneous infusion.

23. (canceled)

24. (canceled)


SEQUENCE LISTING (53 sequences)

SEQ ID NO: 1 - protein, 70 aa, Homo Sapiens
GPETLCGAELVDALQFVCGDRGFYFNKPTGYGSSSRRAPQTGIVDECCFRSCDLRRLEMYCAPLKPAKSA

SEQ ID NO: 2 - protein, 35 aa, Homo Sapiens
RSVRAQRHTDMPKTQKEVHLKNASRGSAGNKNYRM

SEQ ID NO: 3 - protein, 77 aa, Homo Sapiens
RSVRAQRHTDMPKTQKYQPPSTNKNTKSQRRKGWPKTHPGGEQKEGTEASLQIRGKKKEQRREIGSRNAECRGKKGK

SEQ ID NO: 4 - protein, 40 aa, Homo Sapiens
RSVRAQRHTDMPKTQKYQPPSTNKNTKSQRRKGSTFEERK

SEQ ID NO: 5 - protein, 105 aa, Homo Sapiens
GPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 6 - protein, 102 aa, Artificial Sequence
  hIGF1-Ea-mut 03
GPTLCGAELVDALQFVCGDRGFYFNKPTGYGSSSRAAPQTGIVDECCFRSCDLRRLEMYCAPLKPAKSAVRAQRHTDMPKTQKEVHLKNASRGSAGNKNYRM

SEQ ID NO: 7 - DNA, 306 bp, Artificial Sequence
  DNA encoding the hIGF1-Ea-mut 03
ggaccgacgctctgcggggctgagctggtggatgctcttcagttcgtgtgtggagacaggggcttttatttcaacaagcccacagggtatggctccagcagtcgggcggcgcctcagacaggcatcgtggatgagtgctgcttccggagctgtgatctaaggaggctggagatgtattgcgcacccctcaagcctgccaagtcagctgtccgtgcccagcgccacaccgacatgcccaagacccagaaggaagtacatttgaagaacgcaagtagagggagtgcaggaaacaagaactacaggatg

SEQ ID NO: 8 - protein, 102 aa, Artificial Sequence
  mutated human IGF-1 precursor protein
GPTLCGAELVDALQFVCGDRGFYFNKPTGYGSSSQRAPQTGIVDECCFRSCDLRRLEMYCAPLKPAKSAVRAQRHTDMPKTQKEVHLKNASRGSAGNKNYRM

SEQ ID NO: 9 - protein, 102 aa, Artificial Sequence
  mutated human IGF-1 precursor protein
GPTLCGAELVDALQFVCGDRGFYFNKPTGYGSSSREAPQTGIVDECCFRSCDLRRLEMYCAPLKPAKSAVRAQRHTDMPKTQKEVHLKNASRGSAGNKNYRM

SEQ ID NO: 10 - protein, 102 aa, Artificial Sequence; identical to SEQ ID NO: 6
  mutated human IGF-1 precursor protein

SEQ ID NO: 11 - protein, 102 aa, Artificial Sequence
  mutated human IGF-1 precursor protein
GPTLCGAELVDALQFVCGDRGFYFNKPTGYGSSSRPAPQTGIVDECCFRSCDLRRLEMYCAPLKPAKSAVRAQRHTDMPKTQKEVHLKNASRGSAGNKNYRM

SEQ ID NO: 12 - protein, 102 aa, Artificial Sequence
  mutated human IGF-1 precursor protein
GPTLCGAELVDALQFVCGDRGFYFNKPTGYGSSSQQAPQTGIVDECCFRSCDLRRLEMYCAPLKPAKSAVRAQRHTDMPKTQKEVHLKNASRGSAGNKNYRM

SEQ ID NO: 13 - protein, 102 aa, Artificial Sequence
  mutated human IGF-1 precursor protein
GPTLCGAELVDALQFVCGDRGFYFNKPTGYGSSSQAAPQTGIVDECCFRSCDLRRLEMYCAPLKPAKSAVRAQRHTDMPKTQKEVHLKNASRGSAGNKNYRM

SEQ ID NO: 14 - protein, 102 aa, Artificial Sequence
  mutated human IGF-1 precursor protein
GPTLCGAELVDALQFVCGDRGFYFNKPTGYGSSSQRAPQTGIVDECCFRSCDLRRLEMYCAPLKPAKSAVQAQRHTDMPKTQKEVHLKNASRGSAGNKNYRM

SEQ ID NO: 15 - protein, 102 aa, Artificial Sequence
  mutated human IGF-1 precursor protein
GPTLCGAELVDALQFVCGDRGFYFNKPTGYGSSSQRAPQTGIVDECCFRSCDLRRLEMYCAPLKPAKSAVQAQQHTDMPKTQKEVHLKNASRGSAGNKNYRM

SEQ ID NO: 16 - protein, 102 aa, Artificial Sequence
  mutated human IGF-1 precursor protein
GPTLCGAELVDALQFVCGDRGFYFNKPTGYGSSSQRAPQTGIVDECCFRSCDLRRLEMYCAPLKPAKSAVQAQQHTDMPKTQKEVHLKNASRGSAGNKNYQM

SEQ ID NO: 17 - protein, 102 aa, Artificial Sequence
  mutated human IGF-1 precursor protein
GPTLCGAELVDALQFVCGDRGFYFNKPTGYGSSSREAPQTGIVDECCFRSCDLRRLEMYCAPLKPAKSAVRAQQHTDMPKTQKEVHLKNASRGSAGNKNYRM

SEQ ID NO: 18 - protein, 102 aa, Artificial Sequence
  mutated human IGF-1 precursor protein
GPTLCGAELVDALQFVCGDRGFYFNKPTGYGSSSREAPQTGIVDECCFRSCDLRRLEMYCAPLKPAKSAVQAQQHTDMPKTQKEVHLKNASRGSAGNKNYRM

SEQ ID NO: 19 - protein, 102 aa, Artificial Seqeunce
  mutated human IGF-1 precursor protein
GPTLCGAELVDALQFVCGDRGFYFNKPTGYGSSSREAPQTGIVDECCFRSCDLRRLEMYCAPLKPAKSAVQAQQHTDMPKTQKEVHLKNASRGSAGNKNYQM

SEQ ID NO: 20 - protein, 102 aa, Artificial Sequence
  mutated human IGF-1 precursor protein
GPTLCGAELVDALQFVCGDRGFYFNKPTGYGSSSRAAPQTGIVDECCFRSCDLRRLEMYCAPLKPAKSAVQAQRHTDMPKTQKEVHLKNASRGSAGNKNYRM

SEQ ID NO: 21 - protein, 102 aa, Artificial Sequence
  mutated human IGF-1 precursor protein
GPTLCGAELVDALQFVCGDRGFYFNKPTGYGSSSRAAPQTGIVDECCFRSCDLRRLEMYCAPLKPAKSAVQAQQHTDMPKTQKEVHLKNASRGSAGNKNYRM

SEQ ID NO: 22 - protein, 102 aa, Artificial Sequence
  mutated human IGF-1 precursor protein
GPTLCGAELVDALQFVCGDRGFYFNKPTGYGSSSRAAPQTGIVDECCFRSCDLRRLEMYCAPLKPAKSAVQAQQHTDMPKTQKEVHLKNASRGSAGNKNYQM

SEQ ID NO: 23 - protein, 102 aa, Artificial Sequence
  mutated human IGF-1 precursor protein
GPTLCGAELVDALQFVCGDRGFYFNKPTGYGSSSRPAPQTGIVDECCFRSCDLRRLEMYCAPLKPAKSAVQAQRHTDMPKTQKEVHLKNASRGSAGNKNYRM

SEQ ID NO: 24 - protein, 102 aa, Artificial Sequence
  mutated human IGF-1 precursor protein
GPTLCGAELVDALQFVCGDRGFYFNKPTGYGSSSRPAPQTGIVDECCFRSCDLRRLEMYCAPLKPAKSAVQAQQHTDMPKTQKEVHLKNASRGSAGNKNYRM

SEQ ID NO: 25 - protein, 102 aa, Artificial Sequence
  mutated human IGF-1 precursor protein
GPTLCGAELVDALQFVCGDRGFYFNKPTGYGSSSRPAPQTGIVDECCFRSCDLRRLEMYCAPLKPAKSAVQAQQHTDMPKTQKEVHLKNASRGSAGNKNYQM

SEQ ID NO: 26 - protein, 102 aa, Artificial Sequence
  mutated human IGF-1 precursor protein
GPTLCGAELVDALQFVCGDRGFYFNKPTGYGSSSQQAPQTGIVDECCFRSCDLRRLEMYCAPLKPAKSAVQAQRHTDMPKTQKEVHLKNASRGSAGNKNYRM

SEQ ID NO: 27 - protein, 102 aa, Artificial Sequence
  mutated human IGF-1 precursor protein
GPTLCGAELVDALQFVCGDRGFYFNKPTGYGSSSQQAPQTGIVDECCFRSCDLRRLEMYCAPLKPAKSAVQAQQHTDMPKTQKEVHLKNASRGSAGNKNYRM

SEQ ID NO: 28 - protein, 102 aa, Artificial Sequence
  mutated human IGF-1 precursor protein
GPTLCGAELVDALQFVCGDRGFYFNKPTGYGSSSQAAPQTGIVDECCFRSCDLRRLEMYCAPLKPAKSAVQAQQHTDMPKTQKEVHLKNASRGSAGNKNYRM

SEQ ID NO: 29 - protein, 102 aa, Artificial Sequence
  mutated human IGF-1 precursor protein
GPTLCGAELVDALQFVCGDRGFYFNKPTGYGSSSQQAPQTGIVDECCFRSCDLRRLEMYCAPLKPAKSAVQAQQHTDMPKTQKEVHLKNASRGSAGNKNYQM

SEQ ID NO: 30 - protein, 102 aa, Artificial Sequence
  mutated human IGF-1 precursor protein
GPTLCGAELVDALQFVCGDRGFYFNKPTGYGSSSQAAPQTGIVDECCFRSCDLRRLEMYCAPLKPAKSAVQAQQHTDMPKTQKEVHLKNASRGSAGNKNYQM

SEQ ID NO: 31 - protein, 99 aa, Artificial Sequence
  mutated human IGF-1 precursor protein
GPTLCGAELVDALQFVCGDRGFYFNKPTGYGSSSQRAPQTGIVDECCFRSCDLRRLEMYCAPLKPAVRAQRHTDMPKTQKEVHLKNASRGSAGNKNYRM

SEQ ID NO: 32 - protein, 99 aa, Artificial Sequence
  mutated human IGF-1 precursor protein
GPTLCGAELVDALQFVCGDRGFYFNKPTGYGSSSREAPQTGIVDECCFRSCDLRRLEMYCAPLKPAVRAQRHTDMPKTQKEVHLKNASRGSAGNKNYRM

SEQ ID NO: 33 - protein, 99 aa, Artificial Sequence
  mutated human IGF-1 precursor protein
GPTLCGAELVDALQFVCGDRGFYFNKPTGYGSSSRAAPQTGIVDECCFRSCDLRRLEMYCAPLKPAVRAQRHTDMPKTQKEVHLKNASRGSAGNKNYRM

SEQ ID NO: 34 - protein, 99 aa, Artificial Sequence
  mutated human IGF-1 precursor protein
GPTLCGAELVDALQFVCGDRGFYFNKPTGYGSSSRPAPQTGIVDECCFRSCDLRRLEMYCAPLKPAVRAQRHTDMPKTQKEVHLKNASRGSAGNKNYRM

SEQ ID NO: 35 - protein, 100 aa, Artificial Sequence
  mutated human IGF-1 precursor protein
GPTLCGAELVDALQFVCGDRGFYFNKPTGYGSSSQQAPQTGIVDECCFRSCDLRRLEMYCAPLKPAQVRAQRHTDMPKTQKEVHLKNASRGSAGNKNYRM

SEQ ID NO: 36 - protein, 99 aa, Artificial Sequence
  mutated human IGF-1 precursor protein
GPTLCGAELVDALQFVCGDRGFYFNKPTGYGSSSQAAPQTGIVDECCFRSCDLRRLEMYCAPLKPAVRAQRHTDMPKTQKEVHLKNASRGSAGNKNYRM

SEQ ID NO: 37 - protein, 99 aa, Artificial Sequence
  mutated human IGF-1 precursor protein
GPTLCGAELVDALQFVCGDRGFYFNKPTGYGSSSQRAPQTGIVDECCFRSCDLRRLEMYCAPLKPAVQAQRHTDMPKTQKEVHLKNASRGSAGNKNYRM

SEQ ID NO: 38 - protein, 99 aa, Artificial Sequence
  mutated human IGF-1 precursor protein
GPTLCGAELVDALQFVCGDRGFYFNKPTGYGSSSQRAPQTGIVDECCFRSCDLRRLEMYCAPLKPAVQAQQHTDMPKTQKEVHLKNASRGSAGNKNYRM

SEQ ID NO: 39 - protein, 99 aa, Artificial Sequence
  mutated human IGF-1 precursor protein
GPTLCGAELVDALQFVCGDRGFYFNKPTGYGSSSQRAPQTGIVDECCFRSCDLRRLEMYCAPLKPAVQAQQHTDMPKTQKEVHLKNASRGSAGNKNYQM

SEQ ID NO: 40 - protein, 99 aa, Artificial Sequence
  mutated human IGF-1 precursor protein
GPTLCGAELVDALQFVCGDRGFYFNKPTGYGSSSREAPQTGIVDECCFRSCDLRRLEMYCAPLKPAVQAQRHTDMPKTQKEVHLKNASRGSAGNKNYRM

SEQ ID NO: 41 - protein, 99 aa, Artificial Sequence
  mutated human IGF-1 precursor protein
GPTLCGAELVDALQFVCGDRGFYFNKPTGYGSSSREAPQTGIVDECCFRSCDLRRLEMYCAPLKPAVQAQQHTDMPKTQKEVHLKNASRGSAGNKNYRM

SEQ ID NO: 42 - protein, 99 aa, Artificial Sequence
  mutated human IGF-1 precursor protein
GPTLCGAELVDALQFVCGDRGFYFNKPTGYGSSSREAPQTGIVDECCFRSCDLRRLEMYCAPLKPAVQAQQHTDMPKTQKEVHLKNASRGSAGNKNYQM

SEQ ID NO: 43 - protein, 99 aa, Artificial Sequence
  mutated human IGF-1 precursor protein
GPTLCGAELVDALQFVCGDRGFYFNKPTGYGSSSRAAPQTGIVDECCFRSCDLRRLEMYCAPLKPAVQAQRHTDMPKTQKEVHLKNASRGSAGNKNYRM

SEQ ID NO: 44 - protein, 99 aa, Artificial Sequence
  mutated human IGF-1 precursor protein
GPTLCGAELVDALQFVCGDRGFYFNKPTGYGSSSRAAPQTGIVDECCFRSCDLRRLEMYCAPLKPAVQAQQHTDMPKTQKEVHLKNASRGSAGNKNYRM

SEQ ID NO: 45 - protein, 99 aa, Artificial Sequence
  mutated human IGF-1 precursor protein
GPTLCGAELVDALQFVCGDRGFYFNKPTGYGSSSRAAPQTGIVDECCFRSCDLRRLEMYCAPLKPAVQAQQHTDMPKTQKEVHLKNASRGSAGNKNYQM

SEQ ID NO: 46 - protein, 99 aa, Artificial Sequence
  mutated human IGF-1 precursor protein
GPTLCGAELVDALQFVCGDRGFYFNKPTGYGSSSRPAPQTGIVDECCFRSCDLRRLEMYCAPLKPAVQAQRHTDMPKTQKEVHLKNASRGSAGNKNYRM

SEQ ID NO: 47 - protein, 99 aa, Artificial Sequence
  mutated human IGF-1 precursor protein
GPTLCGAELVDALQFVCGDRGFYFNKPTGYGSSSRPAPQTGIVDECCFRSCDLRRLEMYCAPLKPAVQAQQHTDMPKTQKEVHLKNASRGSAGNKNYRM

SEQ ID NO: 48 - protein, 99 aa, Artificial Sequence
  mutated human IGF-1 precursor protein
GPTLCGAELVDALQFVCGDRGFYFNKPTGYGSSSRPAPQTGIVDECCFRSCDLRRLEMYCAPLKPAVQAQQHTDMPKTQKEVHLKNASRGSAGNKNYQM

SEQ ID NO: 49 - protein, 99 aa, Artificial Sequence
  mutated human IGF-1 precursor protein
GPTLCGAELVDALQFVCGDRGFYFNKPTGYGSSSQQAPQTGIVDECCFRSCDLRRLEMYCAPLKPAVQAQRHTDMPKTQKEVHLKNASRGSAGNKNYRM

SEQ ID NO: 50 - protein, 99 aa, Artificial Sequence
  mutated human IGF-1 precursor protein
GPTLCGAELVDALQFVCGDRGFYFNKPTGYGSSSQQAPQTGIVDECCFRSCDLRRLEMYCAPLKPAVQAQQHTDMPKTQKEVHLKNASRGSAGNKNYRM

SEQ ID NO: 51 - protein, 99 aa, Artificial Sequence
  mutated human IGF-1 precursor protein
GPTLCGAELVDALQFVCGDRGFYFNKPTGYGSSSQAAPQTGIVDECCFRSCDLRRLEMYCAPLKPAVQAQQHTDMPKTQKEVHLKNASRGSAGNKNYRM

SEQ ID NO: 52 - protein, 99 aa, Artificial Sequence
  mutated human IGF-1 precursor protein
GPTLCGAELVDALQFVCGDRGFYFNKPTGYGSSSQQAPQTGIVDECCFRSCDLRRLEMYCAPLKPAVQAQQHTDMPKTQKEVHLKNASRGSAGNKNYQM

SEQ ID NO: 53 - protein, 99 aa, Artificial Sequence
  mutated human IGF-1 precursor protein
GPTLCGAELVDALQFVCGDRGFYFNKPTGYGSSSQAAPQTGIVDECCFRSCDLRRLEMYCAPLKPAVQAQQHTDMPKTQKEVHLKNASRGSAGNKNYQM